(19) United States
(12) Patent Application Publication
(10) Pub. No.: US 2015/0094327 A1
(43) Pub. Date: Apr. 2, 2015

(54) COMPOSITIONS AND METHODS FOR INHIBITING RESOLVASES

(71) Applicant: The Trustees of the University of Pennsylvania, Phildalphia, PA (US)

(72) Inventors: Frederic D. Bushman, Rose Valley, PA (US); Young Hwang, Cherry Hill, NJ (US); Matt Culyba, Philadelphia, PA (US)

(21) Appl. No.: 14/397,994

(22) PCT Filed: Apr. 29, 2013
(86) PCT No.: PCT/US13/38650

§ 371 Date: Oct. 30, 2014

Related U.S. Application Data
(60) Provisional application No. 61/640,928, filed on May 1, 2012.

(51) Int. Cl. C07D 471/04; C12Q 1/44

(52) U.S. Cl. CPC C07D 471/04; C12Q 1/44; G01N 2333/065; G01N 2333/916; G01N 2500/02
USPC 514/300; 546/123; 435/238; 435/19; 435/6.1; 506/11

(57) ABSTRACT

The invention provides a fluorescence polarization (FP)-based assay to identify inhibitors of resolvase's DNA cleavage activity. The invention also provides resolvase inhibitors identified by the assay, as well as derivatives and analogs thereof. In certain embodiments, the compounds of the invention are useful to treat a poxvirus infection in an infected subject.

### CROSS-REFERENCE TO RELATED APPLICATION

This application claims priority to U.S. Provisional Application Ser. No. 63/640,928, filed May 1, 2012, the contents of which is incorporated by reference herein in its entirety.

### BACKGROUND OF THE INVENTION

Holliday junction resolvases cleave DNA four-way junctions (Holliday junctions) to yield two free duplex DNAs. For poxviruses, resolvase activity is required to complete synthesis of the viral genomic DNA (Garcia, et al., 2000, Proc Natl Acad Sci USA 97:8926-8931; Garcia, et al., 2001, J Virol 75:6460-6471; Culyba, et al., 2006, Virology 352:466-476; Culyba, et al., 2007, J Biol Chem 282, 34644-34652; Culyba, et al., 2010, J Mol Biol 399 (1):82-95; Culyba, et al., 2009, J Biol Chem 284:1190-1201). The early steps of poxvirus DNA replication result in the production of concatemeric arrays of viral DNA (Moss, B. & Fields., B. N., 2001, Virology. Lippincott-Raven, Philadelphia, pp. 2637-2672). The viral DNA at concatemer junctions can fold to make Holliday junction-like structures, which are cleaved to yield monomeric genomic DNAs. The reaction is required for poxvirus replication (Garcia, et al., 2000, Proc Natl Acad Sci USA 97:8926-8931; Garcia, et al., 2001, J Virol 75:6460-6471).

Poxvirus resolvase is a member of the RNase H superfamily of enzymes, which also includes HIV integrase. Members of this enzyme family have a similar protein fold in their catalytic domains and likely share related catalytic mechanisms to carry out their respective nucleotidyl phosphotransfer reactions (Nowotny, et al., 2005, Cell 121:1005-1016; Lovell, et al., 2002, Nat Struct Biol 9:278-281; Yang and Steitz, 1995, Structure 3:131-134). Each enzyme is dependent on divalent metal ions for activity and contains three or four conserved acidic residues that bind metal ions in the active site. Structural studies of several family members reveal two metal binding sites at the active site, suggesting a conserved two metal-ion catalytic mechanism. Biochemical evidence have been reported to demonstrate that poxvirus resolvase also has an active site that binds metal (Culyba, et al., 2010, J Mol Biol 399 (2):382-95).

Small molecule Inhibitors that target HIV integrase have been developed for the treatment of HIV infection, and one such inhibitor, raltegravir, has been approved by the FDA (Hazuda, et al., 2000, Science 287:646-650; Summa, et al., 2008, J Med Chem 51 (18):5843-55). These inhibitors contain a metal chelating pharmacophore believed to disrupt catalysis by binding divalent metal-ion cofactors at the enzyme active site (Grobler, et al., 2002, Proc Natl Acad Sci USA 99:6661-6666; Hare, et al., 2010, Nature 464:232-236).

Initial studies of poxvirus resolvase have focused on the version of the enzyme encoded by vaccinia virus. This is of interest as an inhibitor target because it differs from the variola (smallpox) enzyme by only two amino acid substitutions, and variola virus may be used as a bioweapon. However, it has been found that the purified vaccinia enzyme is insoluble and is not suitable for high throughput screening (Garcia, et al., 2000, Proc Natl Acad Sci USA 97:8926-8931; Garcia, et al., 2001, J Virol 75:6460-6471; Culyba, et al, 2006, Virology 352:466-476; Culyba, et al., 2007, J Biol Chem 282, 34644-34652; Culyba, et al., 2010, J Mol Biol 399 (1):182-95; Culyba, et al., 2009, J Biol Chem 284:1190-1201). Recently, the purified fowlpox virus resolvase was characterized and found to be much more tractable in biochemical assays and well suited for high throughput screening (Culyba, et al., 2009, J Biol Chem 284:1190-1201; Culyba, et al., 2010, J Mol Biol 399 (1):182-95). The fowlpox resolvase shares only 43% amino acid identity with the vaccinia resolvase, though sequence alignments suggest the active sites are similar.

Resolvase enzymes that cleave DNA four-way (Holliday) junctions are required for poxvirus replication, but clinically useful inhibitors have not been developed. There is thus a need in the art for identifying and generating inhibitors that can be used clinically to treat a poxvirus infection. The present invention addresses this unmet need in the art.

### SUMMARY OF THE INVENTION

The invention provides a composition comprising at least one compound of formula (I), or a salt, solvate, or N-oxide thereof:

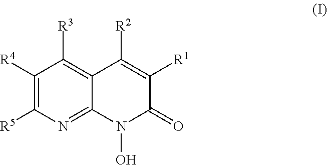

wherein:

each occurrence of R¹ is independently selected from the group consisting of H, —C₁-C₆ alkyl, —C₁-C₆ alkenyl, —C₁-C₆ fluoroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), C₁-C₄ alkenyl-(aryl), and C₁-C₄ alkenyl-(heteroaryl), F, Cl, Br, I, —CN, —NO₂, —OR⁶, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;
wherein the alkyl, alkenyl, aryl, heteroaryl, heteroalkyl, cycloalkyl or heterocycloalkyl group is optionally substituted with 0-5 substituents, each of which is independently selected from the group consisting of —C₁-C₆ alkyl, —C₁-C₆ alkenyl, —C₁-C₆ fluoroalkyl, aryl, heteroaryl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), F, Cl, Br, I, —CN, —NO₂, —OR6, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;

each occurrence of R² is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alky-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ and R² combine to form a (C₃-C₇)heterocycloalkyl group, a (C₃-C₇)cycloalkyl group, a (C₃-C₇)aryl group, or a (C₃-C₇)heteroaryl group optionally substituted with 0-2 R¹ groups;

each occurrence of R³, R⁴, and R⁵ is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O))N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₈ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R² groups;
alternatively, R³ and R⁴ are combined to form a (C₃-C₇)heterocycloalkyl group, a (C₃-C₇)cycloalkyl group, a (C₃-C₇)aryl group, or a (C₅-C₇)heteroaryl group optionally substituted with 0-2 R¹ groups; and,
each occurrence of R⁶ is independently selected from the group consisting of H, C₁-C₆ alkyl, C₁-C₆ ; heteroalkyl, and —C₁-C₂ alkyl-(C₃-C₆ cycloalkyl), wherein the alkyl, heteroalkyl, or cycloalkyl group is optionally substituted with 0-5 R¹ groups.
In one embodiment, the in formula (I) R² is —OH and R³ and R³ are H.
In one embodiment, the formula (I) R¹ is —C(O)OCH₂CH, R² is —OH, and R¹ and R² are H.
In one embodiment, the compound of formula (I) is selected from the group consisting of:
6-([1,1′-biphenyl]-3-yl)-1,4-dihydroxy-3-phenyl-1,8-naphthyridin-2(1H)-one;
1,4-dihydroxy-3-phenyl-6(4-trifluoromethyl)phenethyl)-1,8-naphthyridin-2(1H)-one;
1,4-dihydroxy-6-(4-methoxyphenethyl)-3-phenyl-1,8-naphthyridin-2(1H)-one;
ethyl 1,4-dihydroxy-2-oxo-6-(4-(trifluoromethyl)phenethyl)-1,2-dihydro-1,8-naphthyridine-3-carboxylate;
ethyl 1,4-dihydroxy-6-(2-(6-methoxypyridin-2-yl)ethyl)-2-oxo-1,2-dihydro-1,8-naphthyridine-3-carboxylate;
   a salt or solvate thereof, and any combinations thereof.

In one embodiment, the composition further comprises a pharmaceutically acceptable carrier.

The invention also provides a method of inhibiting poxvirus replication in a subject in need thereof, comprising administering to the subject at least one compound of formula (I), or a salt, solvate, or N-oxide thereof:

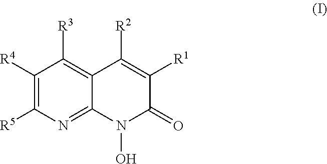

wherein:

each occurrence of R¹ is independently selected from the group consisting of H, —C₁-C₆ alkyl, —C₁-C₆ alkenyl, —C₁-C₆ fluoroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), C₁-C₄ alkenyl-(aryl), and C₁-C₄ alkenyl-(heteroaryl), F, Cl, Br, I, —CN, —NO₂, —OR⁶, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;
wherein the alkyl, alkenyl, aryl, heteroaryl, heteroalkyl, cycloalkyl or heterocycloalkyl group is optionally substituted with 0-5 substituents, each of which is independently selected from the group consisting of —C₁-C₆ alkyl, —C₁-C₆ alkenyl, —C₁-C₆ fluoroalkyl, aryl, heteroaryl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), F, Cl, Br, I, —CN, —NO₂, —OR6, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;

each occurrence of R² is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alky-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ and R² combine to form a (C₃-C₇)heterocycloalkyl group, a (C₃-C₇)cycloalkyl group, a (C₃-C₇)aryl group, or a (C₃-C₇)heteroaryl group optionally substituted with 0-2 R¹ groups;

each occurrence of R³, R⁴, and R⁵ is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O))N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups;
alternatively, R³ and R⁴ are combined to form a (C₃-C₇)heterocycloalkyl group, a (C₃-C₇)cycloalkyl group, a (C₃-C₇)aryl group, or a (C₅-C₇)heteroaryl group optionally substituted with 0-2 R¹ groups; and,
each occurrence of R⁶ is independently selected from the group consisting of H, C₁-C₆ alkyl, C₁-C₆ ; heteroalkyl, and —C₁-C₃ alkyl-(C₃-C₆ cycloalkyl), wherein the alkyl, heteroalkyl, or cycloalkyl group is optionally substituted with 0-5 R¹ groups.

In one embodiment, the subject is a mammal. In one embodiment, the mammal is a human.

The invention also provides a method of inhibiting poxvirus growth, the method comprising contacting the poxvirus with a growth inhibitory amount of at least one compound of formula (I), or a salt, solvate, or N-oxide thereof:

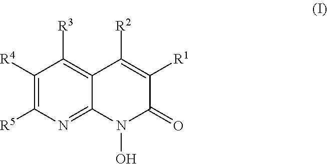

wherein:

each occurrence of R¹ is independently selected from the group consisting of H, —C₁-C₆ alkyl, —C₁-C₆ alkenyl, —C₁-C₆ fluoroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), C₁-C₄ alkenyl-(aryl), and C₁-C₄ alkenyl-(heteroaryl), F, Cl, Br, I, —CN, —NO₂, —OR⁶, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;
wherein the alkyl, alkenyl, aryl, heteroaryl, heteroalkyl, cycloalkyl or heterocycloalkyl group is optionally substituted with 0-5 substituents, each of which is independently selected from the group consisting of —C₁-C₆ alkyl, —C₁-C₆ alkenyl, —C₁-C₆ fluoroalkyl, aryl, heteroaryl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), F, Cl, Br, I, —CN, —NO₂, —OR6, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;

each occurrence of R² is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alky-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ and R² combine to form a (C₃-C₇)heterocycloalkyl group, a (C₃-C₇)cycloalkyl group, a (C₃-C₇)aryl group, or a (C₃-C₇)heteroaryl group optionally substituted with 0-2 R¹ groups;
each occurrence of R³, R⁴, and R⁵ is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O))N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups;
alternatively, R³ and R⁴ are combined to form a (C₃-C₇)heterocycloalkyl group, a (C₃-C₇)cycloalkyl group, a (C₃-C₇)aryl group, or a (C₅-C₇)heteroaryl group optionally substituted with 0-2 R¹ groups; and,
each occurrence of R⁶ is independently selected from the group consisting of H, C₁-C₆ alkyl, C₁-C₆ ; heteroalkyl, and —C₁-C₂ alkyl-(C₃-C₆ cycloalkyl), wherein the alkyl, heteroalkyl, or cycloalkyl group is optionally substituted with 0-5 R¹ groups.
The invention also provides a method of treating a poxvirus infection in a subject in need thereof, the method comprising administering to the subject an effective amount of at least one compound of formula (I), or a salt, solvate, or N-oxide thereof:

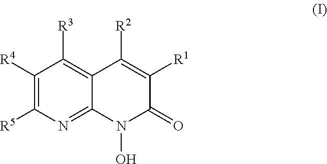

wherein:

each occurrence of R¹ is independently selected from the group consisting of H, —C₁-C₆ alkyl, —C₁-C₆ alkenyl, —C₁-C₆ fluoroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), C₁-C₄ alkenyl-(aryl), and C₁-C₄ alkenyl-(heteroaryl), F, Cl, Br, I, —CN, —NO₂, —OR⁶, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;

wherein the alkyl, alkenyl, aryl, heteroaryl, heteroalkyl, cycloalkyl or heterocycloalkyl group is optionally substituted with 0-5 substituents, each of which is independently selected from the group consisting of —C₁-C₆ alkyl, —C₁-C₆ alkenyl, —C₁-C₆ fluoroalkyl, aryl, heteroaryl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), F, Cl, Br, I, —CN, —NO₂, —OR6, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;

each occurrence of R² is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alky-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ and R² combine to form a (C₃-C₇)heterocycloalkyl group, a (C₃-C₇)cycloalkyl group, a (C₃-C₇)aryl group, or a (C₃-C₇)heteroaryl group optionally substituted with 0-2 R¹ groups;
each occurrence of R³, R⁴, and R⁵ is independently selected from the group consisting of H, —OR⁶, —C(═O)R⁶, —OC(═O)R⁶, —CO₂R⁶, —OCO₂R⁶, —CH(R⁶)₂, —N(R⁶)₂, C(═O)N(R⁶)₂, —OC(═O)N(R⁶)₂, —NHC(═O)NH(R⁶), —NHC(═O)R⁶, —NHC(═O)OR⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alky-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ groups;
alternatively, R³ and R⁴ are combined to form a (C₃-C₇)heterocycloalkyl group, a (C₃-C₇)cycloalkyl group, a (C₃-C₇)aryl group, or a (C₅-C₇)heteroaryl group optionally substituted with 0-2 R¹ groups; and,
each occurrence of R⁶ is independently selected from the group consisting of H, C₁-C₆ alkyl, C₁-C₆ ; heteroalkyl, and —C₁-C₂ alkyl-(C₃-C₆ cycloalkyl), wherein the alkyl, heteroalkyl, or cycloalkyl group is optionally substituted with 0-5 R¹ groups.

The invention also provides a method of identifying a modulator of resolvase, said method comprising: (a) incubating a test substance in the presence of a protein having resolvase activity and a substrate; and (b) determining the effect of the test substance on resolvase activity, wherein a change in resolvase activity as compared to a control means that the test substance is a modulator of resolvase activity.

In one embodiment, the protein having resolvase activity is resolvase

In one embodiment, the substrate is labeled.

In one embodiment, the substrate is a nucleic acid molecule that represents a Holliday junction.

In one embodiment, the substrate is an off-center bulged nucleic acid molecule.

In one embodiment, the resolvase activity is the ability to cleave the substrate, wherein detection of cleavage is measured by characterizing the size of the cleaved substrate.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A through 1G, is a series of images showing results from Holliday junction cleavage by poxvirus resolvase and DNA substrates studied. FIG. 1A is an image demonstrating the role of resolvase in poxvirus DNA replication. The initial product of DNA replication resembles a linear concatemer of many genomes, though in reality the structure is likely branched. The structure to the right emphasizes the Holliday junction formed by refolding of inverted repeats at the viral termini (DNA from the coordinate file 1FLO). FIG. 1B is an image demonstrating cleavage of a Holliday junction substrate tracked by FP. FIG. 1C is an image demonstrating cleavage of a Y-DNA substrate tracked by FP. FIG. 1D is an image demonstrating lack of cleavage of a single DNA strand (3′ labeled). FIG. 1E is an image demonstrating lack of cleavage of a single DNA strand (5′ labeled). FIG. 1F is an image demonstrating lack of cleavage of a duplex DNA. FIG. 1G is an image demonstrating efficient cleavage of an off-center DNA bulge substrate. Sequences of oligonucleotide substrates are in Table 1.

FIG. 2, comprising FIGS. 2A through 2C, is a series of images depicting cleavage of the bulged DNA substrate by fowlpox resolvase. FIG. 2A is an image depicting analysis of cleavage products by native gel electrophoresis. Products were characterized by comigration with synthetic markers identical to each expected product. FIG. 2B is an image depicting analysis of products of cleavage of the bulged DNA substrate analyzed by denaturing gel electrophoresis. The inferred sites of cleavage are shown at the bottom. “Mix” indicates a mixture of the synthetic 15 nt expected cleavage product and an authentic reaction mixture, indicating comigration with the indicated product. FIG. 2C is an image demonstrating that resolvase catalytic site mutants obstruct resolvase cleavage as measured in the FP assay. The bulged DNA substrate was mixed with D7A, D135A, or wild-type resolvase, then cleavage tracked for the indicated times.

FIG. 3 is an image of the 1-hydroxynaphthrydinene backbone, showing the potential metal binding pharmacophore (shaded). For synthetic methods see FIGS. 7 and 8.

FIG. 4 is an image demonstrating inhibition of cleavage of a Holliday junction substrate by fowlpox resolvase in the presence of compound 7. Holliday junction substrates were fluorescently labeled on one DNA strand. Reaction products were analyzed after separation by electrophoresis on a native polyacrylamide gel.

FIG. 5, comprising FIGS. 5A and 5B, is a series of images showing a Michaelis-Menten analysis of cleavage by fowlpox resolvase. FIG. 5A is an image showing selected fits to kinetic plots of reaction progression profiles at different concentrations of substrate. FIG. 5B is an image showing plot of rates at different substrate concentrations.

FIG. 6 is an image an image demonstrating lack of inhibition of variola topoisomerase activity by compound 7. Supercoiled DNA was exposed to purified variola topoisomerase in vitro. Products were then separated by electrophoresis on a native agarose gel and stained with ethidium bromide. The relaxation products (upper bands) were not reduced in intensity by the presence of compound 7.

FIG. 7 illustrates the general synthetic scheme used to prepare non-limiting examples of compounds contemplated within the invention.

FIG. 8 illustrates the synthetic scheme for preparation of 1-hydroxy-1,8-naphthyridin-2(1H)-one compounds.

### DETAILED DESCRIPTION

The present invention relates to the unexpected discovery that an assay based on fluorescence polarization (FP) may be used for high-throughput screening and mechanistic studies to evaluate resolvase cleavage activity. Using this assay, 1-hydroxy-1,8-naphthyridin-2(1H)-one compounds were identified as inhibitors of resolvase cleavage activity. Structure activity (SAR) studies revealed functional parallels to FDA-approved drugs targeting the related HIV integrate enzyme. In some non-limiting instances, 1-hydroxy-1,8-naphthyridin-2(1H)-one compounds exhibited anti-poxvirus activity.

In one embodiment, the invention provides an assay for identifying a compound that inhibits resolvase cleavage activity. In one embodiment, the assay comprises a resolvase substrate wherein the substrate is a nucleic acid molecule comprising a DNA bulge. Preferably, the resolvase substrate is labeled with a detectable marker.

The invention also provides compounds identified by the assay of the invention. In one embodiment, the compounds of the invention bind to and modulate the activity of resolvase. These compounds are useful in the treatment of resolvase-related diseases and disorders, either alone or in combination with at least one additional therapeutic agent, in one embodiment, the resolvase modulator of the invention is art antagonist, inverse agonist or agonist of resolvase.

In one embodiment, the compounds of the invention may inhibit resolvase activity and/or viral (e.g., poxvirus) growth. Among other things, these compounds may be used to treat, viral (such as poxvirus) infection, such as smallpox and a variety of other human and veterinary diseases. Pharmaceutically acceptable salts and stereoisomers of the compounds also are contemplated in some embodiments.

The present invention also provides a method for inhibiting poxvirus replication in a subject. The method comprises administering to the subject a composition of the Invention by any suitable route of administration. For example, the method of the present invention is useful for administrating the compositions of the invention to a subject exposed to a poxvirus including, but not limited to, the smallpox virus, and to a subject who is at risk of contact with the poxvirus.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About,” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate lo perform the disclosed methods.

The term “abnormal,” when used in the context of organisms, tissues, cells or components thereof, refers to those organisms, tissues, cells or components thereof that differ in at least one observable or delectable characteristic (e.g., age, treatment, time of day, etc) from those organisms, tissues, cells or components thereof that display the “normal” (expected) respective characteristic. Characteristics that are normal or expected for one cell or tissue type might be abnormal for a different cell or tissue type.

As used herein, the term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art, it should be understood that the instructions for use of the pharmaceutical composition may be contained on the packaging containing the pharmaceutical composition, and as such the instructions form an increased functional relationship to tire packaged product. However, it should be understood that the instructions may contain information pertaining to the compound's ability to perform its intended function, e.g., treating or preventing a disease in a subject.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

The term “host”, as used heroin, unless otherwise specified, includes mammals (e.g., cats, dogs, horses, mice, etc.), humans, or other organisms in need of treatment. The host is for example, a human or an animal, including without limitation, primates, including macaques, and baboons, as wells as chimpanzee, gorilla, and orangutan; ruminants, including sheep, goats, deer, and cattle, for example, cows, steers, bulb, and oxen; swine, including pigs; and poultry including chickens, turkeys, ducks, and geese.

The terms “individual,” “host,” “subject,” and “patient” are used interchangeably herein, and refer to a mammal, including, but not limited to, primates, including simians and humans.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “poxvirus” is any virus belonging to the family Poxviridae. Poxviridae are characterized by, at least, a relatively large, double-stranded DNA genome (ranging from approximately 130 to 400 kbp). Virions are enveloped, slightly pleomorphic, ovoid, or brick shaped (approximately 140-260 nm in diameter and 220-450 nm long). Virions are composed of an external coat containing lipid and tubular or globular protein structures enclosing one or two lateral bodies and a core, which contains the genome. Particular poxviruses may belong to the chordopoxyirinae or ontomopoxyirinae subfamily, which infect vertebrate or insect hosts, respectively. A poxvirus of the chordopoxyirinae subfamily may further belong to the genus Orthopoxvirus (including, e.g., monkeypox virus, vaccinia virus, buffalopox virus, camelpox virus, cowpox virus, elephantpox virus, variola virus (such as variola major and/or variola minor viruses), volepox virus, ectromelia virus, raccoonpox virus, skunkpox virus, or taterapox virus), Parapoxvirus (including, e.g., bovine papular stomatitis virus, Orf virus, psuedocowpox virus, sealpox virus, or Auzduk disease virus), Avipoxvirus (including, e.g., fowlpox virus), Capripoxvirus (including, e.g., sheeppox virus, lumpy skin disease virus, or goatpox virus). Leporipoxvirus (including, e.g., myxoma virus, or Shope fibroma virus), Suipoxvirus (including, e.g., swinepox virus), Mollusciposvirus (including, e.g., molluscum contagiosum virus), or Yatapoxvirus (including, e.g., tanapox virus or Yaba monkey tumor virus). Viruses of the Othropoxvirus and Parapoxvirus genera can be further characterized as zoonotic (including, e.g., monkeypox virus, vaccinia virus, buffalopox virus, camelpox virus, cowpox virus, elephantpox virus, bovine papular stomatitis virus, Orf virus, psuedocowpox virus, or sealpox virus) or nonzoonotic (including, e.g., variola virus, volepox virus, octromelia virus, raccoonpox virus, skunkpox virus, taterapox virus, or Auzduk disease virus). Zoonotic viruses can infect multiple species of hosts (e.g., humans and animals), while nonzoonotic viruses are believed to infect only a single host species (e.g., humans, fowl, or monkey, etc.). In some examples, a poxvirus is an Orthopoxvirus. In more specific examples, a poxvirus is vaccinia virus or variola virus. The term “poxvirus.” further includes naturally-occurring (e.g., wild-type) poxvirus; naturally-occurring poxvirus variants; and poxvirus variants generated in the laboratory, including variants generated by selection, variants generated by chemical modification, and genetically modified variants (e.g., poxvirus modified in a laboratory by recombinant DNA methods),

As used herein, the term “pharmaceutical composition” or “composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability, “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corm starch and potato starch; ellulose, and its derivatives, such as sodium carboxymethyl cellulose, ethyl cellulose and cellulose acetate; powdered tragacanth; malt; gelatin; talc; recipients, such as cocoa butter and suppository waxes; oils, such as peanut oil, cottonseed oil, safflower oil, sesame oil, olive oil, com oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

A “therapeutic” treatment is a treatment administered to a subject who exhibits signs or symptoms of pathology, for the purpose of diminishing or eliminating those signs or symptoms.

As used herein, “treating a disease or disorder” means reducing the frequency or severity with which a sign or symptom of the disease or disorder is experienced by a patient.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

An “effective amount” of a delivery vehicle is that amount sufficient to effectively bind or deliver a compound.

As used herein, the term “potency” refers to the dose needed to produce half the maximal response (ED₅₀).

As used herein, the term “efficacy” refers to the maximal effect (Eₘₐₓ) achieved within an assay.

As used herein, the term “Pd(dppf)Cl₂” refers to 1,1′-bis(diphenylphosphino)ferrocene]dichloropalladium, or any salt or solvate thereof.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alky is include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with art aromatic ring. Cycloalkyl groups include groups having from 3 to 10 ring atoms. Illustrative examples of cycloalkyl groups include, but are not limited to, the following moieties:

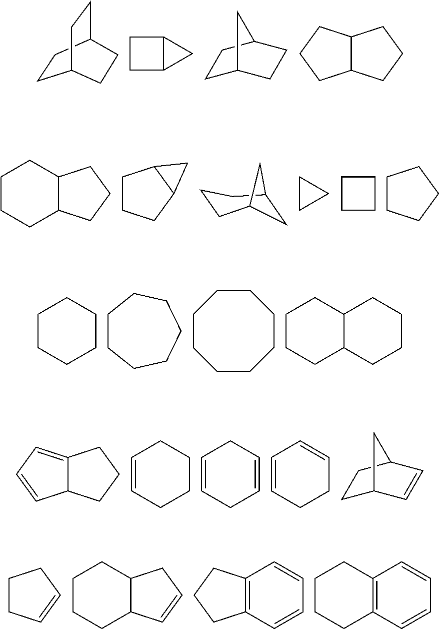

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

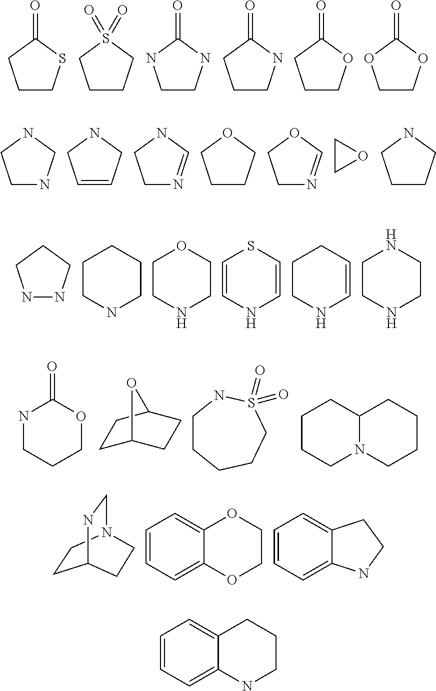

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. Preferred examples are phenyl and naphthyl, most preferred is phenyl.

As used herein, the term “aryl-(C₁-C₃)” means a functional group wherein a one- to three-carbon alkylene chain is attached to an aryl group, e.g., —CH₂CH₂-phenyl. Preferred is aryl-CH₂— and aryl-CH(CH₃)—. The term “substituted aryl-(C₁C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heteroaryl” or “heteroaromatic” refers to a heterocyclic having aromatic character. A polycyclic heteroaryl may include one or more rings that are partially saturated. Examples include the following moieties:

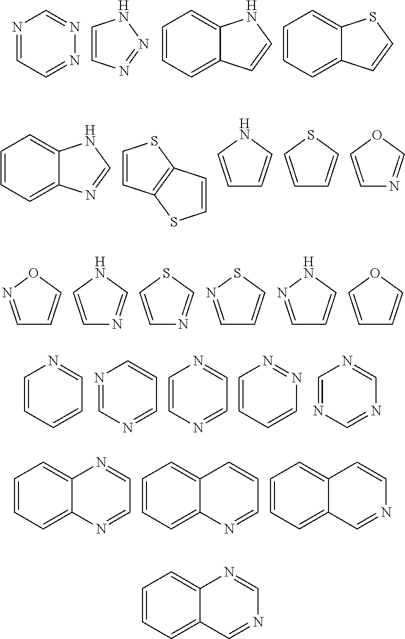

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 3,3,4-oxadiazolyl. Examples of polycyclic heterocycles and heteroaryls include indonyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom, or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)MH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆alkyl, —OH, C₁₋₆ alkoxy, halo, amino, acetamido, oxo and nitro. In yet another embodiment, the substituents are independently selected from the group consisting of C₁₋₆alkyl C₁₋₆ alkoxy, halo, acetamido, and nitro. As used herein, where a substituent is an alkyl or alkoxy group, the carbon chain may be branched, straight or cyclic, with straight being preferred.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as irons 1 to 3, from 1 to 4, from 1 to 5, from 2 to 4, from 2 to 6, from 3 to 6 etc, as well as individual numbers within that range, for example, 1, 2, 2.7, 3, 4, 5, 5.3, and 6. This applies regardless of the breadth of the range.

### DESCRIPTION

The present invention is based on the discovery that a fluorescence polarization (FP) assay may be used for high-throughput screening and mechanistic studies to evaluate resolvase cleavage activity. In one embodiment, the assay of the invention may be used to identify inhibitors of resolvase. An example of an inhibitor identified by the methods of the invention in 1-hydroxy-1,8-naphthyridin-2(1H)-one compounds.

Accordingly, the invention provides compositions and methods for identifying inhibitors of resolvase. In addition, the invention includes compounds identified by the screening methods of the invention. The identified compounds, including derivatives, analogs, and equivalents thereof, may be used to treat poxvirus infection.

In one embodiment, the compounds of the invention are useful, at least, in the treatment of poxvirus infection (such as smallpox) and to inhibit virus (such as poxvirus growth). In sonic instances, the compounds of the invention exert an inhibitory effect on poxviruses (and thereby treat pox virus-related disease) by interfering with the activity of resolvase. In some instances, inhibition of resolvase specifically targets viral replication.

### Screening Assay

The invention provides a screening assay to identify modulators of resolvase activity. Preferably, the resolvase activity being modulated is the ability for the resolvase to cleave a substrate. In one embodiment, the substrate is a nucleic acid molecule. In another embodiment, the substrate is a nucleic acid that represents a Holliday junction.

In one embodiment, the assay is based on using fluorescence polarization (FP) for monitoring DNA cleavage by resolvase. FP relies on the anisotropic properties of the light emitted from fluorophores tumbling in solution after excitation with polarized light. Therefore, the substrate for resolvase activity can be tagged with a detectable label. Preferably, the detectable label is a fluorescein-label, Therefore, in one embodiment, to perform the assay, a solution containing a fluorescently tagged molecule is exposed to a pulse of plane polarized light and the polarization of the emitted light is measured in two orthogonal planes simultaneously. Large molecules rotate more slowly in solution and, when tagged with a fiuorophore, the light emitted is more polarized than for a small molecule. Thus, if the fluorescently tagged substrate and product differ significantly in size, the reaction progress can be monitored by FP.

Accordingly, the present invention provides a method of identifying a modulator of resolvase comprising; (a) incubating a test substance in the presence of resolvase and a substrate; and (b) determining the effect of the test substance on resolvase activity, wherein a change it) resolvase activity as compared to a control means that the test substance is a modulator of resolvase activity. In one embodiment, the resolvase activity is the ability to cleave the substrate wherein detection of cleavage is measured by characterizing the size of the detectable substrate. For example, active resolvase cleaves the substrate to yield at least two cleavage products, The cleaved products may be detected by any method in the art (e.g, electrophoresis). A test compound able to inhibit the ability of resolvase to cleave the substrate is considered an inhibitor of resolvase.

The phrase “determining the effect of the test substance on resolvase activity” means that the effect of the test substance on the activity of resolvase will be assayed and compared to the activity that is normally observed in the absence of the test substance. Preferably the screening assay is repeated using a control sample with the same conditions and components as the test sample but without the test substance. The activity of resolvase in the presence of the test substance is then directly compared to the control.

In one embodiment, a test compound able to inhibit the ability of resolvase to cleave the substrate is considered an inhibitor of resolvase. Thus, a preferred embodiment of the invention includes an assay for identifying inhibitors of resolvase. The term “inhibitor of resolvase” or “resolvase inhibitor” means any substance or agent that causes a decrease in, or inhibition of, resolvase activity as compared to the activity in the absence of the substance or agent.

The resolvase enzyme used in the assay may be in purified or isolated form such as recombinant resolvase. Alternatively, cells that produce resolvase may be used as the resolvase source for the assay.

In one embodiment, the resolvase substrate is a nucleic acid molecule. In another embodiment, the substrate comprises a ten-nucleotide bulge region flanked on one side by a long region of duplex DNA and on the other side by a 5 bp duplex region, or stem (e.g., “asymmetric bulge”).

In a further embodiment, the resolvase substrate may also comprise a label, for example a fluorescent or radioactive label, which may be used to monitor tire progress of the resolvase cleavage reaction.

The activity of resolvase may be assayed by monitoring the appearance of the expected DNA product from the action of resolvase on the resolvase substrate. Any known method for detecting nucleic acid molecules may be used to monitor the appearance of the expected product. For example, fluorescent substrates or radioactive substrates may be used and the products assayed using standard methods. Other forms of electrophoresis (e.g., capillary electrophoresis) or other technologies, such as mass spectrometry, a fluorescence reading or other methods to monitor resolvase activity are similarly included within the scope of the present application.

Accordingly, in an embodiment of the present invention, there is provided a method of identifying a modulator of resolvase comprising: (a) incubating a test substance in the presence of resolvase and a resolvase substrate; and (b) assaying for the presence of an expected product; wherein a change in an amount of expected product in the presence of the test substance compared to a control indicates that the test substance is a modulator of resolvase. By “control” it is meant performing the method using the same conditions and components as with the test substance, but without the test substance.

In a further embodiment of the present invention, the resolvase substrate is attached to a solid support. Attaching the substrate to a solid support is especially useful in high-throughput screening for modulators of resolvase, for example, an oligonucleotide substrate may be bound to the wells (384 or greater) of streptavidin coated assay plates. The oligonucleotide substrate may contain a label, for example a biotin label, at one end to tether the substrate to the streptavidin coated wells of the assay plate and another label, for example a fluorescent or radioactive label, at the other end for detection purposes. The action of resolvase on this substrate will sever the fluorescent or radioactive label from the portion of the substrate which is linked to the plate. The fluorescent or radioactive label would then be removable from the well by a simple washing step. Determination of fluorescence using a fluorescence micro-plate reader or radioactivity using standard counters allows a facile assay for resolvase activity. When the enzyme is active, the fluorescence or radioactivity will decrease after incubation with resolvase. If the reaction is blocked by an inhibitor the fluorescence or radioactivity level will remain constant or show substantially less reduction than in the absence of inhibitor (control).

The lest substance can be any compound which one wishes to test including, but not limited to, proteins (including antibodies), peptides, nucleic acids (including RNA, DNA, antisense oligonucleotide, peptide nucleic acids), carbohydrates, organic compounds, inorganic compounds, natural products, library extracts, bodily fluids and other samples that one wishes to test for modulators of resolvase. More than one test compound can be tested at a time in the assay of the invention. As such the assay is useful in testing the combined effects of two or more compounds on the modulation of resolvase.

As discussed elsewhere herein, the method is adaptable to high-throughput screening applications. For example, a high-throughput screening assay may be used, comprising any of the methods according to the invention wherein aliquots of resolvase and corresponding substrate are exposed to a plurality of test compounds within different wells of a multi-well plate. Further, a high-throughput screening assay according to the invention involves aliquots of resolvase and corresponding substrate which are exposed to a plurality of candidate substances in a miniaturized assay system of any kind. Another embodiment of a high-throughput screening assay could involve exposing aliquots of resolvase and corresponding substrate simultaneously to a plurality of test compounds.

The method of the invention may be “miniaturized” in an assay system through any acceptable method of miniaturization, including but not limited to multi-well plates, such as 24, 48, 96 or 384-wells per plate, micro-chips or slides. The assay may be reduced in size to be conducted on a micro-chip support, advantageously involving smaller amounts of reagents and other materials. Any miniaturization of the process which is conducive to high-throughput screening is within the scope of the invention.

In a specific embodiment, the screening assay is used to identify inhibitors of resolvase. The screening assays of the invention are useful in identifying resolvase inhibitors that are useful in identifying potential therapeutic or agents for treating poxvirus infections.

### Compounds of the Invention

The compounds of the present invention may be synthesized using techniques well-known in the art of organic synthesis. The starting materials and intermediates required for the synthesis may be obtained from commercial sources or synthesized according to methods known to those skilled in the art.

In one aspect, the compound of the invention is a compound of formula (I), or a salt, solvate, or N-oxide thereof:

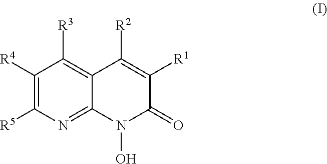

wherein:

each occurrence of R¹ is independently selected from the group consisting of H, —C₁-C₆ alkyl, —C₁-C₆ alkenyl, —C₁-C₆ fluoroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), C₁-C₄ alkenyl-(aryl), and C₁-C₄ alkenyl-(heteroaryl), F, Cl, Br, I, —CN, —NO₂, —OR⁶, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;

wherein the alkyl, alkenyl, aryl, heteroaryl, heteroalkyl, cycloalkyl or heterocycloalkyl group is optionally substituted with 0-5 substituents, each of which is independently selected from the group consisting of —C₁-C₆ alkyl, —C₁-C₆ alkenyl, —C₁-C₆ fluoroalkyl, aryl, heteroaryl, —C₃-C₅ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), F, Cl, Br, I, —CN, —NO₂, —OR6, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;

each occurrence of R² is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alky-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ and R² combine to form a (C₃-C₇)heterocycloalkyl group, a (C₃-C₇)cycloalkyl group, a (C₃-C₇)aryl group, or a (C₃-C₇)heteroaryl group optionally substituted with 0-2 R¹ groups;

each occurrence of R³, R⁴, and R⁵ is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O))N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups;

alternatively, R³ and R⁴ are combined to form a (C₃-C₇)heterocycloalkyl group, a (C₃-C₇)cycloalkyl group, a (C₃-C₇)aryl group, or a (C₅-C₇)heteroaryl group optionally substituted with 0-2 R¹ groups;

each occurrence of R⁶ is independently selected from the group consisting of H, C₁-C₆ alkyl, C₁-C₆ ; heteroalkyl, and —C₁-C₃ alkyl-(C₃-C₆ cycloalkyl), wherein the alkyl, heteroalkyl, or cycloalkyl group is optionally substituted with 0-5 R¹ groups.

In one embodiment, R² is —OH. In another embodiment, R³ and R⁵ are H.

In one embodiment, R¹ is —C(O)OCH₂CH₃, R² is —OH, and R³ and R⁵ are H.

In one embodiment, the compound of formula (I) is selected from the group consisting of:
6-([1,1′-biphenyl]-3-yl)-1,4-dihydroxy-3-phenyl-1,8-naphthyridin-2(1H)-one;
1,4-dihydroxy-3-phenyl-6-(4′-(trifluoromethyl)phenethyl)-1,8-naphthyridin-2(1H)-one;
1,4-dihydroxy-6-(4-methoxyphenethyl)-3-phenyl-1,8-naphthyridin-2(1H)-one;
ethyl 1,4-dihydroxy-2-oxo-6-(4-(trifluoromethyl)phenethyl)-1,2-dihydro-1,8-naphthyridine-3-carboxylate;
ethyl 1,4-dihydroxy-6-(2-(6-methoxypyridin-2-yl)ethyl)-2-oxo-1,2-dihydro-1,8-naphthyridine-3-carboxylate;
   a salt or solvate thereof, and any combinations thereof.

### Preparation of the Compounds of the Invention

Compounds of formula (I) may be prepared by the general schemes described herein, using the synthetic method known by those skilled in the art. The following examples illustrate non-limiting embodiments of the invention.

In a non-limiting embodiment, the synthesis of 1-hydroxy-1,8-naphthyridin-2(1H)-one compounds is accomplished in two steps, first by coupling a 1-(benzyloxy)-6-bromo-1,8-naphthyridin-2(1H)-one with a boronic acid, followed by removal of the benzyl protecting group. A non-limiting example of a coupling method includes heating in dimethylformamide with anhydrous potassium carbonate and Pd(dppf)Cl₂ at about 110° C. In a non-limiting embodiment, the removal of the benzyl protecting group to reveal the desired 1-hydroxy-1,8-naphthyridin-2(1H)-one compound is accomplished under reductive conditions. A non-limiting example of a reductive condition includes treatment with 10% palladium in ethanol/tetrahydrofuran (1:1) under a hydrogen atmosphere.

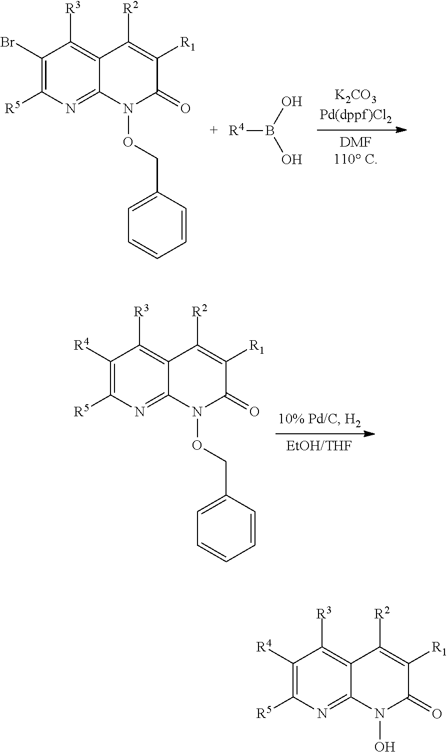

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of coantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds presented herein.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group,

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ¹⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy, isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplemental (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Croups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysis. For example, an allyl-blockcd carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

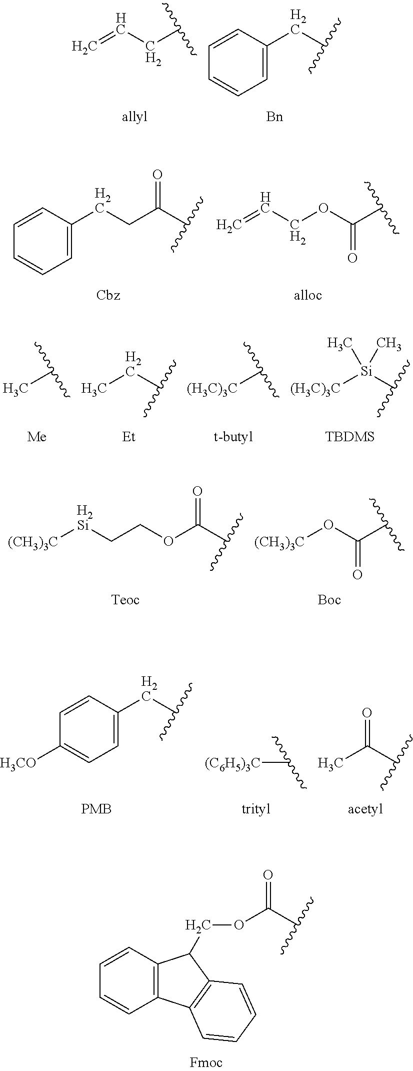

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York. N.Y., 1994, which are incorporated herein by reference for such disclosure.

### Methods

The compositions of the invention are useful, at least, in the treatment of poxvirus infection (such as smallpox) and to inhibit virus (such as poxvirus growth). Such compounds are believed to exert an inhibitory effect on poxviruses (and thereby treat poxvirus-related disease) by interfering with the activity of Resolvase. Accordingly, if it is desirable to do so, non-limiting methods useful to functionally characterize, resolvase inhibitors include virus growth assays (e.g., plaque formation assays in cultured cells and/or virus growth assays in cultured cells). In one embodiment, the compounds of the invention reduce virus growth as compared to such activity or growth measured in the absence of such inhibitor. Numerous assays suitable for the measurement of virus growth, in the presence and absence of disclosed inhibitors are known in the art.

Virus growth assays detect, for example, to what extent (or whether) a putative resolvase inhibitor can slow virus growth. Two exemplary virus growth assays are in vitro cell culture assays and plaque reduction assays.

In a representative in vitro cell culture assay, cultured cells susceptible to infection by the virus of interest (such as a poxvirus, for example vaccinia virus) are grown to a desired cell density under culture conditions suitable to the particular cell type. The cultured ceils then are infected (either with a single or multiple inoculum(s)) with a known amount of virus (such as, about 0.03 plaque-forming units per cell). The virus inoculum remains in contact with the cells for a sufficient time (such as 30 minutes) to permit virus to adsorb to the cells. Unbound virus is removed and medium containing inhibitor or vehicle (control) is then added. Infection is permitted to proceed for a sufficient time (e.g., about 18-24 hours) for the virus to replicate (at least under control conditions) to a comfortably measurable level. Virus is harvested, for example, by removal of the medium (e.g., for viruses that shed from the cells) and/or by collection of infected cells and isolation of virus from such cells.

Virus can be isolated from infected cells by any method known in the art. In one exemplary method, cells are disrupted (e.g., by cycles of freezing and thawing and/or shearing with a syringe needle, such as 1.5 inch 22 gauge needle), and debris is removed by centrifugation. Viruses are collected in the supernatant, which, optionally, is mixed with protease. Debris can be removed by centrifugation. Then, supernatant containing virus is serially diluted for plaque assay.

In the plaque assay, cultured ceils (e.g., in a series of culture dishes) are contacted with virus (e.g., serial dilutions) for a sufficient time to permit virus to adsorb to the cells. The infected monolayers of cells, then, are overlaid with medium containing agarose (and no inhibitor), incubation of the cells is continued for a period of time, after which time the number of plaques is counted. A resolvase inhibitor would be expected to reduce the number of plaques relative to control.

A plaque reduction assay also is useful to determine the ability of a compound to inhibit virus growth (such as, poxvirus growth). In this assay, monolayers of cultured cells susceptible to infection by the virus of interest are exposed to a viral inoculum. After a period of time for adsorption of the viral particles to the cells, the culture medium is removed and replaced with a nutrient agarose containing the test compound, After a period of incubation (e.g., several days), plaques, which are areas where cells have died as a result of viral infection, are counted. In the case of vaccinia virus, plaques can be recognized after about 48 hours by staining with crystal violet or neutral red. An effective resolvase inhibitor would be expected lo reduce the number of plaques as compared to control.

The present invention also includes methods of of treating poxvirus infection and/or inhibiting poxvirus growth. An example of a poxvirus includes, but is not limited to a chordopoxvirus (for example an Orthopoxvirus, Parapoxvirus, Avipoxvirus, Capripoxvirus, Leporipoxvirus, Suipoxvirus, Mollusciposvirus, or Yatapoxvirus, or a combination thereof). Specific method embodiments involve Orthopoxviruses (or resolvases therefrom), including without limitation variola virus, vaccinia virus, monkeypox virus, buffalopox virus, camelpox virus, elephantpox virus, volepox virus, ectromelia virus, raccoonpox virus, skunkpox virus, Uasin Gishu disease virus, or taterapox virus, or a combination thereof. Some method embodiments involve the treatment or growth inhibition of variola virus (such as variola major or variola minor virus), or inhibition of variola virus resolves. Other method embodiments involve poxviruses capable of infecting human hosts (such as monkeypox virus, vaccinia virus, buffalopox virus, cowpox virus, elephantpox virus, variola virus, bovine papular stomatitis virus, orf virus, pseudocowpox virus, sealpox virus, tanapox virus, or Yaba monkey tumor virus, or combinations thereof), or inhibition of resolvases from such human pathogens. Some methods of treatment involve the treatment of smallpox, human monkeypox, parapoxvirus infection, molluseum contagiosum virus infection, or human cowpox.

In certain embodiments, the poxvirus as described herein infects vertebrates. In certain embodiments, the poxvirus as described herein infects invertebrates. In certain embodiments, the poxvirus of the present causes a variety of diseases of veterinary and medical importance. In certain embodiments, the poxvirus as described herein belongs to the chordopoxyirinac subfamily, in another embodiment, the poxvirus as described herein is variola virus (smallpox virus). In another embodiment, the poxvirus is vaccinia virus. In another embodiment, the poxvirus is molluscum contagiosum virus. In other embodiments, the poxvirus is any known orthopoxvirus, parapoxvirus, or yatapoxvirus.

In another embodiment, the poxvirus is a cowpox virus. In another embodiment, the poxvirus is a monkeypox virus. In another embodiment, the poxvirus is a raccoonpox virus. In another embodiment, the poxvirus is a camelpox virus. In another embodiment, the poxvirus is a skunkpox virus. In another embodiment, the poxvirus is a volepox virus. In another embodiment, the poxvirus is an ectromelia virus. In another embodiment, the poxvirus is a taterapox virus.

In another embodiment, the poxvirus is a parapoxvirus. In another embodiment, the poxvirus is an orf virus, in another embodiment, the poxvirus is a pseudocowpox virus. In another embodiment, the poxvirus is any other type of parapoxvirus known in the art.

In another embodiment, the poxvirus is an avipoxvirus. In another embodiment, the poxvirus is a canarypox virus. In another embodiment, the poxvirus is a fowlpox virus. In another embodiment, the poxvirus is any other type of avipoxvirus known in the art.

In another embodiment, the poxvirus is a capripoxvirus. In another embodiment, the poxvirus is a goatpox virus. In another embodiment, the poxvirus is a lumpy skin disease virus. In another embodiment, the poxvirus is any other type of capripoxvirus known in the art.

In another embodiment, the poxvirus is a leporipoxvirus. In another embodiment, the poxvirus is a myxoma virus. In another embodiment, the poxvirus is a fibroma virus. In another embodiment, the poxvirus is any other type of leporipoxvirus known in the art.

In another embodiment, the poxvirus is a molluscipoxvirus. In another embodiment, the poxvirus is a molluseum contagiosum virus. In another embodiment, the poxvirus is any other type of molluscipoxvirus known in the art.

In another embodiment, the poxvirus is a yatapoxvirus. In another embodiment, the poxvirus is a tanapox virus. In another embodiment, the poxvirus is a Yaba monkey tumor virus. In another embodiment, the poxvirus is any other type of yatapoxvirus known in the art.

In another embodiment, the poxvirus is any other type of poxvirus known in the art. In another embodiment, each of the above poxviruses and types of poxviruses represents a separate embodiment of the present invention.

In certain embodiments, methods of inhibiting replication of a poxvirus comprise methods of inhibiting resolvase. In certain embodiments, inhibiting the DNA replication is achieved by inhibiting activity of resolvase.

In one embodiment, the invention provides methods of treating a poxvirus infection or an associated disease include administering a resolvase inhibitor (and, optionally, one or more other pharmaceutical agents) to a subject in a pharmaceutically acceptable carrier and in an amount effective to treat poxvirus infection or an associated disease (such as smallpox). The treatment can be used prophylactically in any subject in a demographic group at substantial risk for such diseases; for example, children in Central Africa (who are at particular risk for monkeypox infection), or persons who have not previously been immunized with a vaccine against poxvirus infection (such as the smallpox vaccine). Alternatively, subjects can be selected using more specific criteria, such as a probable or definitive diagnosis of poxvirus infection or smallpox or other poxvirus-based disease based on, for example, clinical signs and symptoms and/or laboratory evidence of poxvirus infection. For example, smallpox (or variola virus infection) may present clinically with abrupt onset of fever and prostration with a macular rash (on the head, limbs, hands (including palms) and feet (including soles) and inside the mouth), which rash progresses to vesicles which become pustular, ulcerated, scabbed, and healed with scarring; provided that the subject recovers in the face of an approximately 40% mortality rate. Other poxvirus infections may be clinically identified based on localized pustules with scar formation (e.g., vaccinia virus), ulcerative lesions (sometimes called “milkers nodules”; e.g., cowpox); non-ulcerative milker's nodules (e.g., pseudocowpox virus); single painless, papulo-vesicular lesion on the hand, forearm or face (e.g., ORF virus); or other known symptoms of poxvirus infection. Laboratory tests useful for identifying poxvirus infection include histological examination of a curetted or biopsied lesion, electron microscopy, immunohistochemistry using antibodies specific for poxvirus proteins, in situ hybridization or PCR using poxvirus-specific nucleic acid probes or primers, respectively, antigen detecting agar gel immune precipitation test, or other commonly known diagnostic tests (see, e.g., Mangana-Vougiouka et al., Mol. Cell. Probes 14 (5): 305-10, 2000).

The invention also includes methods of inhibiting the growth of a virus (such as a poxvirus, like variola virus) by contacting the virus with a growth inhibitory amount of a resolvase inhibitor of the invention. The phrase “inhibiting virus growth” (and analogous phrases, such as inhibition of virus growth) conveys a wide-range of inhibitory effects that an agent (e.g., resolvase inhibitors) may have on the normal (i.e., control) rate of virus growth, The terms “virus growth,” “Virus multiplication,” and “virus replication” are intended to be synonymous. In some instances, duplication of a viral genome could be used as a measure of virus replication; however, duplication of a viral genome is only one possible (and optional) indicator of viral growth.

The phrase “inhibiting virus growth” (or like terminology) may be considered relative to the normal (i.e., uninhibited or control) rate of growth of a particular virus or viruses of interest (e.g., poxvirus, such as variola virus or vaccinia virus). Thus, inhibiting virus growth includes situations wherein the normal growth rate of a virus has slowed (i.e., virus number (or plaque-forming units (plus)) increases over time, but not as rapidly as control), equals zero (i.e., there is substantially no change in virus number (or plus) in the population over time, e.g., virus growth is approximately equal to inhibition of virus growth), or becomes negative (i.e., virus number (or pfus) decrease over time). A negative rate of virus growth can (but need not) result in clearance of substantially all viruses from a host cell or organism. In particular instances, a resolvase inhibitor can reduce virus growth by at least about 60% (as compared to untreated control); for example, by at least about 70%, by at least about 75%. by at least about 80%, by at least about 85%, by at least about 90%, or even up to by about 95% or nearly 100%.

Contact between a resolvase inhibitor of She invention with a poxvirus may occur in vitro (such as in culture conditions) or in vivo (such as in a subject infected with at least one poxvirus). In certain method embodiments, growth inhibitory amounts include amounts described elsewhere herein (for example, IC₅₀ concentrations). In some examples, a growth inhibitory amount is from about 10 nm to about 1 μM, from about 0.1 μM to about 10 μM, from about 1 μM to about 100 μM, from about 2,5 μM to about 250 μM, or from about 30 μM to about 500 μM (such as front about 25 μM to about 400 μM, from about 50 μM to about 300 μM, or from about 100 μM to about 250 μM).

In one embodiment, the invention includes a method of treating conditions/disease associated with at least one virus in a subject. The method comprises administering to the subject a therapeutically effective amount of a compound of the invention. In some instances, the compounds of the invention are specifically targeted against viral replication and/or virally infected/transformed cells.

In another embodiment, the present invention provides methods of inhibiting, treating, or abrogating a poxvirus infection in a subject; inhibiting replication of a poxvirus; and inhibiting activity of a poxvirus resolvase; comprising contacting a poxvirus with a compound of the present invention.

The present invention further provides methods of prophylactically treating smallpox virus infection. In view of the high mortality rate associated with smallpox virus infection, any expected exposure, suspected exposure, or known exposure to smallpox virus is considered cause for initiating treatment with the methods of the present invention. An advantage of the subject methods is that prophylactic interferon treatment reduces the risk that an individual who has been exposed to smallpox virus will develop an infection with the virus and/or will exhibit clinical symptoms of smallpox virus infection. A further advantage of the subject methods is that prophylactic treatment reduces the clinical symptoms of smallpox virus infection should such an infection occur.

The present invention further provides methods of therapeutically treating smallpox or vaccinia virus infection in an individual who presents clinical signs of smallpox or vaccinia virus infection following known or suspected exposure to smallpox or vaccinia virus or following vaccination with vaccinia virus vaccine, individuals (i) who have received vaccination with vaccinia virus vaccine or who have known or suspected exposure to smallpox or vaccinia virus and (ii) who present a fever often exceeding 40° C. are considered eligible for treatment with the methods of the present invention. An advantage of the present method is that the severity of the smallpox or vaccinia virus infection is reduced, e.g., the viral load is reduced, and/or the time to viral clearance is reduced, and/or the morbidity or mortality is reduced.

Whether a subject treatment method is effective in reducing the risk of a pathological poxvirus infection, reducing viral load, reducing time to viral clearance, or reducing morbidity or mortality due to a poxvirus infection is readily determined by those skilled in the art. Viral load is readily measured by measuring the titer or level of virus in serum. The number of viruses in the serum can be determined using any known assay, including, e.g., a quantitative polymerase chain reaction assay using oligonucleotide primers specific for the poxvirus being assayed. Whether morbidity is reduced may be determined by measuring any symptom associated with a poxvirus infection, including, e.g., fever, the extent of rash formation, the number of pustules, and the like.

Methods of treating, preventing, or ameliorating poxvirus infections are also included in the invention, in practicing the methods, effective amounts of resolvase inhibitor of the invention, or a salt, ester, or prodrug thereof may be administered in any desired manner, e.g., via oral, rectal, nasal, topical (including buccal and sublingual), vaginal, or parenteral (including subcutaneous, intramuscular, subcutaneous, intravenous, intradermal, intraocular, intratracheal, intracisternal, intraperitoneal, and epidural) administration.

### Combination Therapy

A variety of anti-poxvirus compounds may be used in combination with the resolvase inhibitors of the present invention, or its salts, esters or prodrugs thereof, in the methods provided herein. The anti-poxvirus agent may be a pharmaceutical compound, such as a nucleoside or nucleoside analogue, or in another embodiment may be a biologic agent, such as an immunomodulatory amino acid sequence or nucleic acid sequence.

Anti-pox virus compounds that may be used include IMP dehydrogenase inhibitors, such as ribavirin; nucleoside analogues, such as 3′-C-methylcytidine; acyclic nucleoside phosphonates, such as adefovir; 2-, 6- and 8-alkylated adenosine analogues, such as 8-methyladenosine; thiosemicarbazones, such as N-methylisatin 3-thiosemicarbazone. See, e.g. Bray et al., 2003, Antiviral Research 58: 101-134.

Other anti-orthopox virus agents include polyanionic substances (e.g., polyacrylic acid, dextran sulfate, pentosan polysulfate, polyvinyl alcohol sulfate, and polyacrylic acid vinyl alcohol sulfate), N-phosphonoacetyl-L-aspartate, or N¹-isonictinoyl-N²-3-methyl-4-chlorobenzoylhydrazine.

Other additional anti-orthopox agents include for example, a compound used to reduce potential pathology of smallpox vaccination, such as vaccinia immune globin, as well as methisazone, ribavarin, 5-iodo-2,-deoxyuridine, adenine arabinoside, trifluorothymidine, nucleoside analogs and interferon and interferon inducers (see, e.g., Bell et al., 2004, Virology 325:425-431).

The one or more additional anti-poxvirus agent also may be a biologic such as interferon (or an interferon-inducer, such as 4-iodo-antipyrine), pegylated interferon, interferon alpha, beta, gamma, epsilon or tau, interferon alpha 2a, interferon alphacon-1, natural interferon, albuferon, interferon beta-1a, omega interferon, interferon alpha and interferon gamma-1b.

In another embodiment, the resolvase inhibitors of the inventions, or salt, ester or prodrug thereof may be administered in combination or alternation with a biologic agent including immunomodulatory agents, such as colony stimulating factors, e.g. granulocyte macrophage colony-stimulating factor; an interleukin, such as interleukin-1 alpha, interleukin-1 beta, interleukin-2, interleukin-3, interleukin-4, interleukin-5, interleukin-6, interleukin-7, interleukin-8, interleukin-10, interleukin-12; macrophage inflammatory proteins, such as macrophage inflammatory protein-1 alpha, macrophage inflammatory protein-1 beta; and erythropoietin.

Other additional anti-poxvirus agents include cytokines and immunostimulatory sequences (“ISSs”). ISSs are short DMA-like molecules, with distinct nucleotide sequences, that possess potent immunomodulatory properties that direct different immune system functions. ISSs that can be used are described, for example, in U.S. Pat. No. 6.194,388, issued Feb. 27, 2001; U.S. Pat. No. 6,207,646, issued Mar. 27, 2001; and U.S. Pat. No. 6,239,116, issued May 29, 2001, to Coley Pharmaceutical Group et al., the disclosures of which are incorporated herein by reference. Other ISSs that can be used are described in U.S. Pat. No. 6,589,940, issued Jul. 8, 2003; U.S. Pat. No. 6,562,798, issued May 13, 2003; and U.S. Pat. No. 6,225,292, the disclosures of which are incorporated herein by reference. Other useful sequences are described in WO 96/02555; WO 98/18810; WO 98/40100; WO 99/51259; WO 00/06588; U.S. Pat. No. 6,218,371; WO 98/52581; WO 01/22990; WO 01/22972; as well as WO 98/55495; WO 97/28259; WO 98/16247; WO 00/21556; and WO 01/12223, the disclosures of which are incorporated herein by reference.

In another embodiment, the anti-orthopox virus agent may be selected from analogs of adenosine-N(1)-oxide and analogs of 1-(benzyloxy) adenosine, such as 1-(3-methoxybenzyloxy) adenosine and 1-(4-methoxybenzyloxy) adenosine.

In another embodiment, the anti-orthopox virus agents include SAH hydrolase inhibitors, such as 5′-noraristeromycin, neplanocins A and C, carbocyclic 3-deaza-adenosine, 9-(2′,3′-dihyroxycyclopenten-1-yl)adenine, DHCaA, e³DHCeA, C³DHCaA 6′-beta-fluoro-aristeromycin, 5′-noraristeromycin and enantiomers and opimers thereof, 3-deaza-5′-noraristeromycin, 6′-C-methylneplanocin, 6′-homoneplanocin, 2-fluoroneplancin, 6′-iodo acetylenic Ado, and 3-deazaneplanocin.

In one embodiment, the anti-poxvirus agent is selected from one or more of: 8-methyladenosine; 2-amino-7-[(1,3-dihydroxy-2-propoxy)methyl]purine (S2242); (S)-9-(3-hydroxy-2-phosphonylmethoxypropyl)-2,6-diaminopurine ((S)-HPMPDAP); (S)9-(3-hydroxy-2-phosphonylmethoxypropyl)adenine (HPMPA); cyclic HPMPA; 8-Aza-HPMPA; adenine arabinoside; adefovir (PMEA) adefovir dipivoxil; (S)-6-(3-hydroxy-2-phosphonylmethoxy-propyl)oxy-2,4-diammopyrimidine ((S)-HPMPO-DAPy); [(phosphonylmethoxy)ethyl]-N6-(cyclopropyl) DAP (PME-N6-(cyclopropyl) DAP); PME-N6-(dimethyl)DAP; PME-N6-(trifluoroethyl)DAP; PMEA-N6-(2-propenyl)DAP; bis(butyl L-alaninyl) adefovir; bis(butyl) L-alaninyl) PME-N6-(cyclopropyl)DAP; (isopropyl L-alaninyl) phenyl PME-N6-(cyclopropyl)DAP; novobiocin; IMP dehydrogenase inhibitors (e.g., ribavirin, 5-ethynyl-1-beta-D-ribofuranosylimidazole-4-carboxamide (EICAR), FICAR, triazofurin, and selenazole); OMP decarboxylase inhibitors (e.g., pyrazofurin and 5′-deoxypyrazofurin); CTP synthetase inhibitors (e.g., cyclopentenyl cytosine and carbodine); thymidylate sythase inhibitors (e.g., 5-substituted 2′-deoxyuridines), rifampin; and 3′-fluoro-3′-deoxyadenosine.

Optionally, the anti-orthopox virus agents disclosed herein are in the form of a prodrug, including but not limited to the prodrug structures disclosed above. The compounds are, for example, antiviral phosphonate prodrug compounds. Other non-limiting examples of such prodrug structures are described in, for example, U.S. Pat. No. 5,223,263; U.S. Pat. No. 4,619,794; JP Patent 61-152694; U.S. Pat. No. 5,436,234; U.S. Pat. No. 5,411,947; U.S. Pat. No. 5,194,654; U.S. Pat. No. 5,463,092; U.S. Pat. No. 5,512,671; U.S. Pat. No. 5,484,912; U.S. Pat. No. 6,030,960; U.S. Pat. No. 5,962,437; U.S. Pat. No. 6,448,392; U.S. Pat. No. 5,770,584; U.S. Pat. No. 5,869,468; U.S. Pat. No. 5.84,228; U.S. Publication No. 2002/0082242; U.S. Publication No. 2004/0161398; U.S. Publication No. 2004/0259845; WO 98/38202; U.S. Pat. No. 5,696,277; U.S. Pat. No. 6,002,029; U.S. Pat. No. 5,744,592; U.S. Pat. No. 5,827,831; U.S. Pat. No. 5,817,638; and U.S. Pat. No. 6,252,060, the disclosures of which are incorporated herein by reference.

### Administration/Dosage/Formulations

The regimen of administration may affect what constitutes an effective amount. The therapeutic formulations may be administered to the subject either prior to or after the onset of a virus-related disorder or disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat virus-related disorders or diseases in the patient. An effective amount of the therapeutic compound necessary to achieve a therapeutic effect may vary according to factors such as the state of the disease or disorder in the patient; the age, sex, and weight of the patient; and the ability of the therapeutic compound to treat virus-related disorders or diseases in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of virus-related disorders or diseases in a patient.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin. In one embodiment, the pharmaceutically acceptable carrier is not DMSO alone.

In one embodiment, the compositions of the invention are administered to the patient in dosages that range from one to five times per day or more. In another embodiment, the compositions of the invention are administered to the patient in range of dosages that include, but are not limited to, once every day, every two days, every three days to once a week, and once every two weeks. It is readily apparent to one skilled in the art that the frequency of administration of the various combination compositions of the indention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physician taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 rag, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about. 300 mg, or less than about 200 mg, or less than about 100 mg, or less than about 50 mg, or less than about 40 mg, or less than about 30 mg, or less than about 25 mg, or less titan about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using tire compound to treat, prevent, or reduce one or more symptoms of virus-related disorders or diseases in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (transurethral, vaginal (e.g., trans- and perivaginally) (intranasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica), disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa., (e.g., OPADRY™ OY Type, OYC Type, Organic Enteric OY-P Type, Aqueous Enteric OY-A Type, OY-PM Type and OPADRY™ White, 32K 18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient, The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of virus-related diseases or disorders. Using a wax/pH-sensitive polymer mix, a gastric insoluble composition may be obtained in which the active ingredient is entrapped, ensuring its delayed release,

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488.962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and She progression of virus-related disorders or diseases in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”), The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 380 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained, in one embodiment, patients require intermittent treatment on a long-term basis upon any recurrence of symptoms and/or infection.

The compounds for use in the method of the invention may be formulated in unit dosage form. The term “unit, dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Active compounds exhibiting high therapeutic indices are preferred. The data obtained from cell culture assays and animal studies are optionally used in formulating a range of dosage for use in human. The dosage of such active compounds lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLE 1

### Bulged DNA Substrates for Identifying Poxvirus Resolvase Inhibitors

An assay based on fluorescence polarization (FP) was designed to monitor DNA cleavage by fowlpox virus resolvase. FP relies on the anisotropic properties of the light emitted from fluorophores tumbling in solution after excitation with polarized light. To carry out the assay, a solution containing a fluorescently tagged molecule is exposed to a pulse of plane polarized light and the polarization of the emitted light is measured in two orthogonal planes simultaneously. Large molecules rotate more slowly in solution and, when tagged with a fluorophore, the light emitted is more polarized compared to that of a smaller molecule. Thus if the fluorescently tagged substrate and product differ significantly in size, the reaction progress can be monitored by FP.

The disclosure presented herein describes a design of an optimal bulged DNA substrate and its use to screen more than 133,000 small molecules for inhibitory activity. Mapping cleavage sites on the substrate suggested a new activity for the enzyme which may promote efficient DNA replication. The FP assay has excellent high throughput screening parameters, which allowed for the identification of a structural class of inhibitors, 1-hydroxy-1,8-naphthyridin-2(1H)-ones, exhibiting potent activity against fowlpox resolvase. SAR analysis demonstrated a metal-chelating binding mode related to that of raltegravir. Several of these inhibitors also showed antiviral activity against vaccinia virus in cell culture assays.

The materials and methods employed in these experiments are now described.

### Materials and Methods

Purification of Fowlpox Virus Resolvase

Fowlpox virus resolvase was purified as described (Culyba, et al., 2007, J Biol Chem 282:34644-34652; Culyba, et al., 2009, J Biol Chem 284:1190-1201). Briefly, resolvase protein was modified to contain a His-tag. The protein was overexpressed in E. coli and purified by metal affinity chromatography. Protein concentrations were determined by the Bradford assay. The fowlpox resolvase fraction was judged to be >90% pure by SDS-PAGE.

Fluorescence Polarization Substrates

Oligonucleotides containing a 6-carboxyfluorescein end label (F) were purified by high performance liquid chromatography (HPLC). All other oligonucleotides were purified by polyacrylamide gel electrophoresis (PAGE). DNA concentrations were determined by UV-spectrophotometry. The substrates were constructed by annealing together the indicated component oligonucleotides. Annealing reactions contained 10 mM labeled DNA and two fold excess unlabeled DNA and were carried out in the presence of 100 mM NaCl by heating to 95° C. and allowing the solutions to cool slowly to room temperature over a period of 90 minutes.

Cleavage Reactions

For reactions in 384-well plates, reagents were dispensed into wells using automated liquid handlers. Black polystyrene plates coated with a non-binding surface were used (Corning #3575).

For the NSRB-library screen, 20 ml of art enzyme solution or a buffer-only control (i.e. no enzyme) was dispensed into the plate wells. Next, 100 nanoliters of each compound stock solution (5 mg/ml in DMSO) or DMSO was added to wells by robotic pin transfer. Then, 10 ml of a substrate solution was added to achieve a final volume of 30 ml and the plates were incubated at 37° C. for 1 hour. After incubation, fluorescence polarization values were measured using an Envision instrument (Perkin-Elmer). Final reagent concentrations were 2 nM fluorescein-labeled substrate and 10 nM fowlpox resolvase. Final solution conditions were 25 mM Tris-HCl [pH 8.0], 15 mM MgCl, 100 mM NaCl, and 1 mM DTT. Assuming a molecular weight of 500 g/mol, the final compounds concentration was 33 mM.

For the integrase-library screen, 20 ml of an enzyme solution or a buffer-only solution was dispensed into wells containing compound stock solution (in DMSO) or DMSO. Then, 10 ml of a substrate solution was added to achieve a final volume of 30 ml and the plates were incubated at 37° C. for 1 hr. After incubation, fluorescence polarization values were measured using an Analyst instrument (Molecular Devices). Final reagent concentrations and solution conditions were as above. The final concentration of each compound was 20 mM.

The screens were carried out in 384-well plates where each well contained a different compound from the library. The enzyme and compound were dispensed into wells first and then reactions were initiated by addition of the AB5 substrate and incubation at 37° C. After one hour, FP measurements were obtained using a multilabel plate reader. Each plate contained 32 positive control wells (i.e. no enzyme, no inhibitor) and 32 negative control wells (i.e. enzyme, no inhibitor). Library 1 was screened at a compound concentration of 17 mg/ml (33 mM for a compound with molecular weight=500 g/mol) and Library 2 was screened at a compound concentration of 20 mM. The average molecular weight of Library 2 was 416 g/mol (l SD=114), so for comparison to Library 1, the average concentration of Library 2 in mg/ml was 8.3 mg/ml (l SD=2.3), or ˜2-fold less.

Anisotropy of the emitted light is characterized by the polarization, P, and the anisotropy, r, so that P=(V−H)/(V+H) and r=(V−H)/(V+2H), where V and H are the intensities of the emitted light in the vertical and horizontal planes, respectively. The total, fluorescence intensity (TFI) is given by V+2H. Both parameters are related to the rotational velocity of the tagged molecule in solution, which is proportional to its molecular volume.

The TFI measurements were used to identify compounds with either fluorescence enhancing or quenching properties so that potential false positives and negatives can be remove from the analysis. Experiments were designed to first normalize the TFI measurements from each compound-containing well by expressing it as a fraction of the mean TFI of the negative controls located on the same plate. Compound-containing wells with a TFI greater than or less than one standard deviation from the mean TFI of all compound-containing wells were excluded from subsequent analyses. For Library one, 2,234 compounds (1.7%) that met these criteria were identified, of which 98% were fluorescence enhancers. For Library two, 78 compounds (2.8%) that met these criteria were identified, of which 46% were enhancers.

Percent inhibition values for each compound were calculated using the following equation: % inhibition=100*(P−x)/(P−N), where x is the fluorescence polarization value of tire compound well, and P and N are the mean fluorescence polarization values of the positive and negative control wells within the same plate.

For IC₅₀ determination, serial dilutions of the compounds were made in DMSO and the assay was carried out as above. For data analysis, fluorescence polarization values were transformed into anisotropy values using the following equation: r=2P/(3−-P), where r is anisotropy and P is polarization. Percent inhibition values were calculated as above using the transformed data. Non-linear regression was used to fit the data against the logarithm of the compound concentrations using a sigmoidal dose-response model in Prism.

Kinetic Analysis

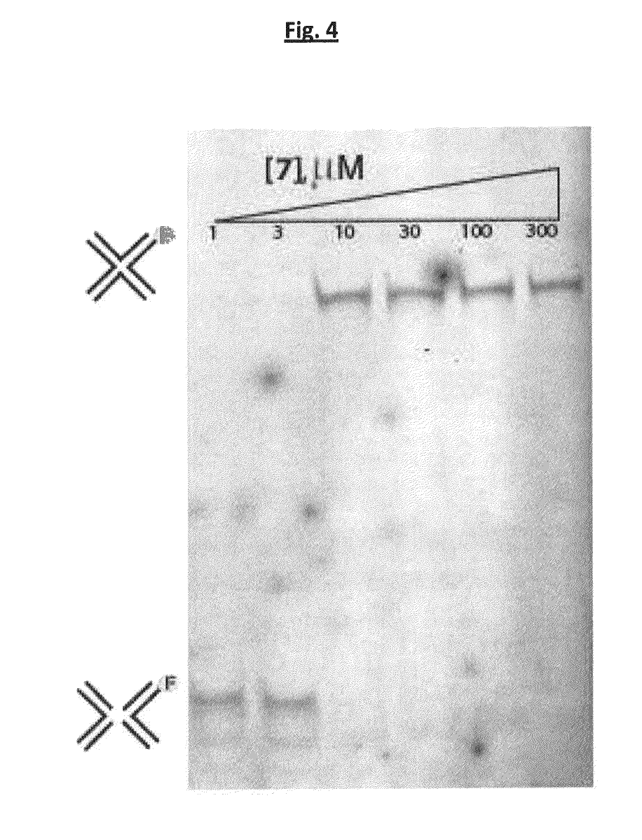

Reactions for kinetic analysis were carried out in 100 ml buffer consisting of 25 mM Tris-Cl pH 8.0, 100 mM NaCl, 15 mM MgCl, and 1 mM DTT at 37° C. The concentration of fowlpox virus resolvase was 10 nM. Fluorescence labeled bulge substrate DNA at a final concentration of 2 nM was mixed with different amounts (0-1,920 nM) of unlabeled bulge substrate DNA. Enzyme solutions were diluted in 2 ml of the reaction buffer and added immediately to the premixed substrate solution before analysis. Positive reference samples contained all components except the enzyme. Cleavage reactions were monitored by measuring change of FP using the Beacon 2000 Fluorescence polarization system (Invitrogen, CA) over a wide range of substrate concentrations (FIG. 5). Initial rates were calculated from non-linear regression using an exponential decay model in Prism. Kₘ and Vₘₐₓ were calculated using the Michaelis-Menten model in Prism.

Antiviral and Cytotoxicity Assays

Plaque assays were performed on confluent BSCl monolayers in 12-well plates. The cells were overlaid with 1 ml of MEM (with 2% FBS) containing approximately 60 PFU of vaccinia virus WR. Plates containing virus-inoculated cells were incubated for 1 hour, the media was then removed and the cells were overlaid with 1 ml of MEM (with 2% FBS) containing serial threefold dilutions of the test compounds. Plaque formation was monitored at 48 hours post-infection by staining monolayers with crystal violet. IC₅₀ was calculated using a sigmoidal dose response model in Prism. DMSO carrier was found to be toxic above −5%.

Cytotoxicity was tested using the CellTiter-Glo Luminescent Cell Viability Assay kit from Promega (Madison, Wis.). Non-linear regression was used to fit the data against the logarithm of the compound concentrations as discussed elsewhere herein.

Preparation of 1-hydroxy-1,8-naphthyridin-2(1H)-one Derivatives

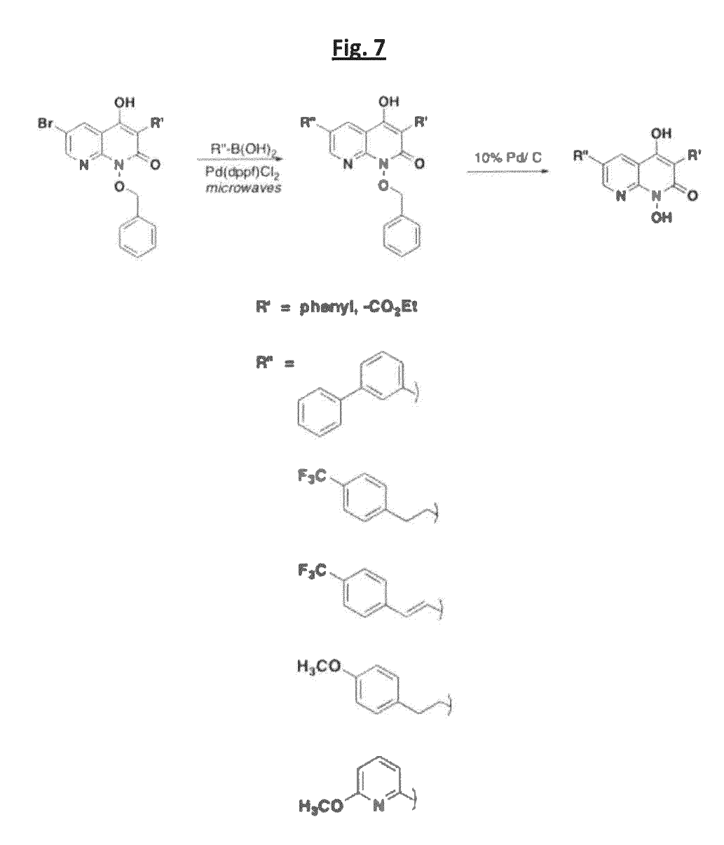

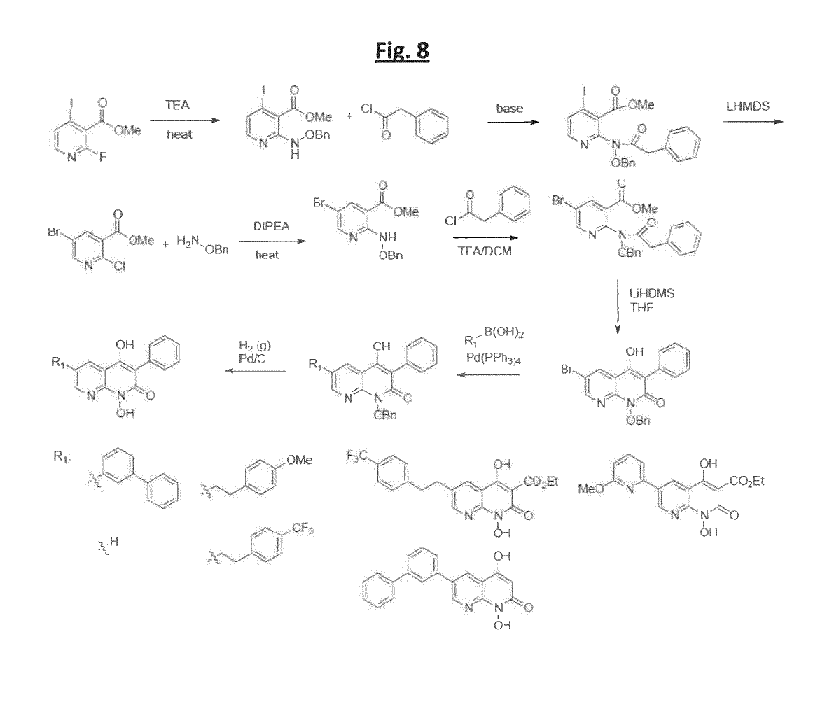

The bromo naphthyridone intermediate was prepared using published procedures (PCT Patent Publ. No. WO 2008/010964 A1) and as described in FIGS. 7 and 8. Commercially available boronic acids were purchased from Sigma Aldrich. Suzuki coupling reactions were used to prepare N-benzyloxynaphthyridone adducts, which were purified by silica gel flash chromatography. The resulting adducts were reduced with H₂/10% Pd on carbon, giving the resulting debenzylated naphthyridone compounds.

General Procedure for Suzuki Couplings

A solution of the 6-bromonaphthyridone intermediate (0.171 mmol) in dimethylformamide (3 ml,) was treated with a boronic acid (0.298 mmol), anhydrous potassium carbonate (54 mg, 0.436 mmol) and water (0.7 mL). The resulting solution was purged with a stream of argon for 10 mm and then treated with Pd(dppf)Cl₂ (18 mg, 0.014 mmol) and heated in a scaled vial using microwave radiation at 110° C. for 20 min. Flash chromatography on silica gel cluting with a gradient of hexane/ethyl acetate (0-10% ethyl acetate in hexanes) gave pure adducts (20-30% yield).

General Procedure for Reduction of N-benzyloxynaphthyridones:

A suspension of N-benzyloxynaphthyridone adducts (0.03 mmol) in ethanol tetrahydrofuran (1:1, 12 mL) was treated with 10% Pd/C (2 mg) and then hydrogenated at 1 atm overnight. The catalyst was removed by filtration through Celite and evaporated in vacuo giving pure products (80-100% yield).

Analytical Data:

6-(Biphenyl-3-yl)-1,4-dihydroxy-3-phenyl-1,8-naphthyridin-2(1/H)-one, Sodium salt (1). ¹H NMR (300 MHz) (CD₃OD) δ 8.86 (d, 1H, J=2.6 Hz), 8.83 (d, 1H, J=2.6 Hz), 7.96 (t, 1H, J=1.65 Hz), 7.73-7.69 (m, 3H), 7.65-7.61 (m, 1H), 7.59-7.54 (m, 3H), 7.50-7.43 (m, 2R), 7.39-7.31 (m, 3H), 7.20-7.14 (m, 1H). MS: m/z (relative intensity) (EST, negative ion) 405 (M−H, 100).

1,4-Dihydroxy-3-phenyl-6-(4-(trifluoromethyl)phenethyl)-1,8-naphthyridin-2(1H)-one (2). ¹H NMR (300 MHz) (CD₃OD) δ 8.36 (s, 1H), 8.25 (s, 1H), 7.22-7.60 (m, 9H), 3.15-3.00 (m, 4H). MS: m/z (relative intensity) (ESI, negative ion) 425 (M−H, 100).

(E)-1-(Benzylyoxy)-4-hydroxy-3-phenyl-6-(4-(trifluoromethyl)styryl-1,8-naphthyridin-2(1H)-one (3), ¹H NMR (300 MHz) (CDCl₃) δ 8.87 (d, 1H, J=2.3 Hz), 8.46 (d, 1H, J=2.3 Hz), 7.74-7.70 (m, 2H), 7.68-7.64 (m, 4H), 7.62-7.49 (m, 5H), 7.40-7.36 (m, 3H), 6.89-6.76 (m, 2H), 5.35 (s, 2H), MS; m/z (relative intensity) (ESI, negative ion) 513 (M−H, 100).

1,4-Dihydroxy-6-(4-methoxyphenethyl)-3-phenyl-1,8-naphthyridin-2(1H)-one(4). ¹H NMR (300 MHz) (CD3OD) δ 8.35 (s, 1H), 8.25 (s, 1H), 3.74 (s, 3H), 3.06-2.91 (m, 4H). MS: m/z (relative intensity) (ESI, negative ion) 387 (M−H, 100).

Ethyl 1,4-dihydroxy-2-oxo-6-(4-(trifluoromethyl)phenethyl)-1,2-dihydro-1,8-naphthyridine-3-carboxylate (5). ¹H NMR (300 MHz) (CD₃OD) δ 8.50 (d, 1H, J=1.7 Hz), 8.35 (d, 1H, J=1.7 Hz), 7.55 (d, 1H, J=8.1 Hz), 7.37 (d, 1H, J=8.1 Hz), 4.47 (q, 2H, J=7.2 Hz), 3.13-3.08 (m, 4H), 1.42 (t, 3H, J=7.2 Hz), MS: m/z (relative intensity) (ESI, negative ion) 421 (M−H, 100).

(E)-Ethyl 1-(benzyloxy)-4-hydroxy-2-oxo-6-(4-(trifluoromethyl)styryl)-1,2-dihydro-1,8-naphthyridine-3-carboxylate (6). ¹H NMR (300 MHz) (CDCl₃) δ 8.90 (d, 1H, J=2.3 Hz). 8.57 (d, 1H, J=2.3 Hz), 7.75-7.69 (m, 2H), 7.68-7.62 (m, 4H), 7.43-7.35 (m, 3H), 7.27-7.21 (m, 3H), 5.30 (s, 2H) 4.56 (q, 2H, J=7.1 Hz), 1.51 (t, 3H, J=7.1 Hz). MS: m/z (relative intensity) (ESI, negative ion) 509 (M−H, 100).

Ethyl 1,4-dihydroxy-6-(6-methoxypyridin-2yl)-2-oxo-1,2-dihydro-1,8-naphthyridine-3-carboxylate (7). ¹H NMR (300 MHz) (CD₃OD) δ 8.99 (s, 1H), 8.72 (s, 1H), 8.50 (s, 1H), 8.15-7.90 (m, 1H), 6.99-6.90 (m, 1H), 4.49 (q, 2H, J=7.1 Hz), 3.97 (s, 3H), 1.43 (t, 3H). MS: m/z (relative intensity) (ESI, negative ion) 356 (M−H, 100).

Ethyl 1-(benzyloxy)-4-hydroxy-6-(6-methoxypyridin-2-yl)-2-oxo-1,2-dihydro-1,8-naphthyridine-3-carboxylate (8). ¹H NMR (300 MHz) (CDCl₃) δ 8.95 (d, 1H, J=2.3 Hz), 8.55 (d, 1H, J=2.3 Hz), 8.44 (d, 1H, J=2.1 Hz), 7.83 (dd, 1H, J=2.1 Hz, J=8.4 Hz), 7.76-7.68 (m, 2H), 7.46-7.36 (m, 3H), 6.89 (d, 1H, J=8.4 Hz), 5.31 (s, 2H), 4.56 (q, 2H, J=6.9 Hz), 4.01 (s, 3H), 1.51 (t, 3H). MS: m/z (relative intensity) (ESI, negative ion)446 (M−H, 100).

The results of the experiments are now described.

Developing a Screening Substrate for Fluorescence Polarization Assays

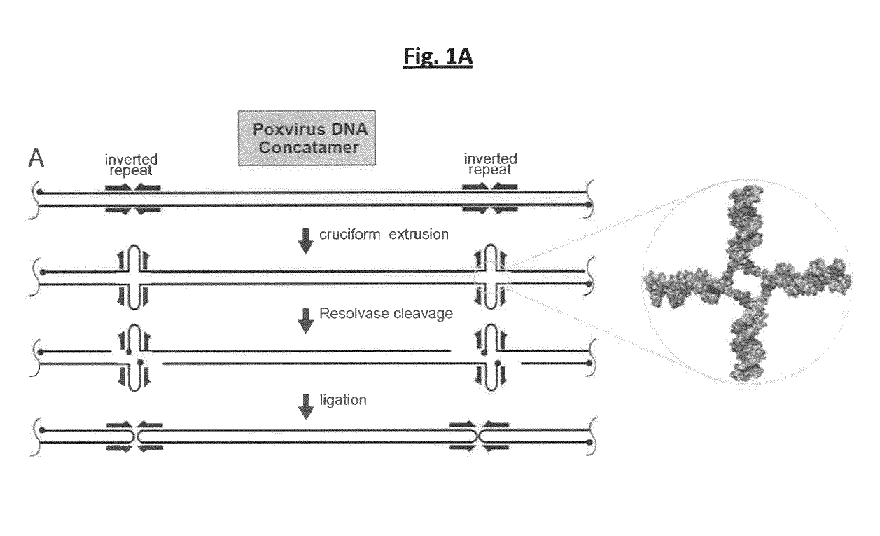

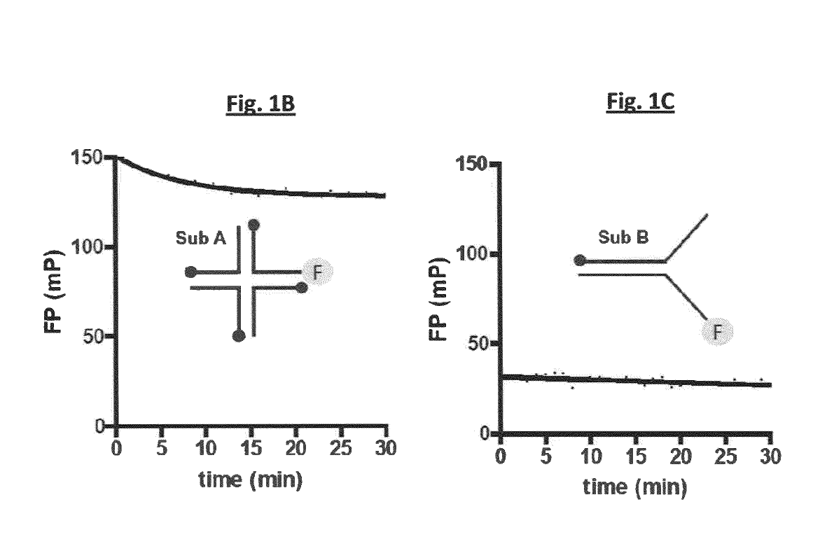

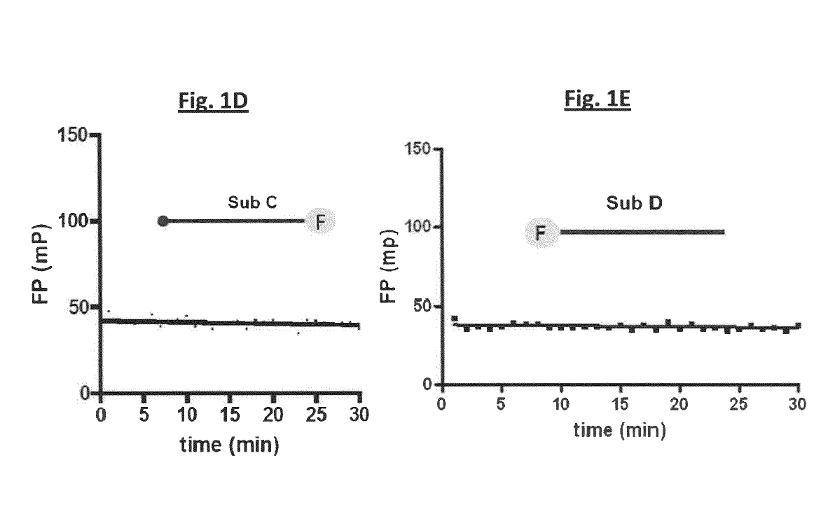

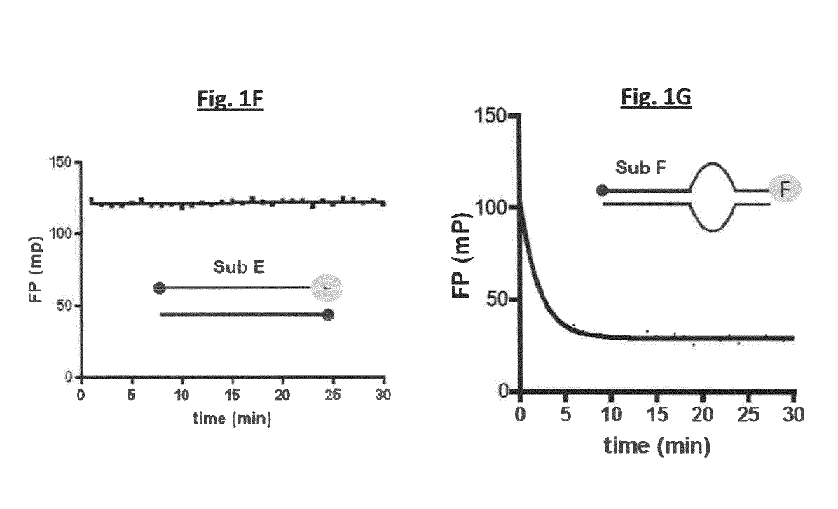

Developing the resolvase substrate for high throughput screening required an iterative series of synthesis and testing steps (FIG. 1; oligonucleotide sequences are in Table 1). Although Holliday junctions represent the physiologic substrate, cleavage of Holliday junctions into nicked duplex products yielded only a slight change in FP (FIG. 1B, sub A), even though gel assays confirmed efficient cleavage. Evidently the molecular weight change was not large enough to yield a significant difference in FP, or the fluorescein group bound to the double stranded DNA end, which would also reduce its mobility and increase polarization. In an effort to increase the dynamic range, a variety of splayed duplex substrates that each contained a single-to-double strand transition was tested (FIG. 1C, sub B). These substrates were fluorescently tagged on the 5′-end single stranded region, so cleavage would yield a short single strand (5 bases for sub B). After incubation with fowlpox resolvase, complete conversion of the substrate to the expected products was confirmed by native polyacrylamide gel electrophoresis. However, sub B exhibited a relatively small change in polarization after incubation with resolvase (ΔP=−10 mP), resulting in a dynamic range that was not adequate for high throughput screening.

TABLE 1
Oligonucleotides used for the assay substrates and size markers

| Substrate | Oligonucleotide sequence | use |
|---|---|---|
| Sub A | 5′-AGCTTCTGTGCAGCAGCTGCTCTCAACTGCAGTCTAGACT-3′; | Holliday |
| | (SEQ ID NO: 1) | Junction |
| | 5′-AGTCTAGACTGCAGTTGAGAGCTTGCTAGGACGGATCCCT-3′; | |
| | (SEQ ID NO: 2) | |
| | 5′-AGGGATCCGTCCTAGCAAGCTTTTTGTTTTGATTGCGAGG-3′; | |
| | (SEQ ID NO: 3) | |
| | 5′-CCTCGCAATCAAAACAAAAAGCAGCTGCTGCACAGAAGCT-3′-F; | |
| | (SEQ ID NO: 4) | |
| Sub B | 5′-CCTCGCAATCAAAACAAAAAGCAGCTGCTGCACAGAAGCT-3; | sprayed |
| | (SEQ ID NO: 5) | duplex |
| | F-5′-GTCGTGCTGCTTTTTGTTTTGATTGCGAGG-3′; | |
| | (SEQ ID NO: 6) | |
| Sub C | 5′-TCCTACCACCAGATACACGCCACAGTTTTTTTTTTGATTA-3′-F; | single |
| | (SEQ ID NO: 7) | stranded |
| Sub D | F-5′-TCCTACCACCAGATACACGCCACAGTTTTTTTTTTGATTA-3′; | single |
| | (SEQ ID NO: 8) | stranded |
| Sub E | 5′-TCCTACCACCAGATACACGCCACAGTTTTTTTTTTGATTA-3′-F; | duplex |
| | (SEQ ID NO: 9) | |
| | 5′-TAATCAAAAAAAAAACTGTGGCGTGTATCTGGTGGTAGGA-3′; | |
| | (SEQ ID NO: 10) | |
| Sub F | 5′-TCCTACCACCAGATACACGCCACAGTTTTTTTTTGATTA-3′-F; | bulged |
| | (SEQ ID NO: 11) | substrate |
| | 5′-TAATCTTTTTTTTTTCTGTGGCGTGATATCTGGTGGTAGGA-3′; | |
| | (SEQ ID NO: 12) | |
| marker | 5′-TCCTACCACCAGATACACGCCACAGTTTTTTTTTTGATTA-3′; | 75 base |
| | (SEQ ID NO: 13) | |
| | 5′ TTTTTTTTTTCTGTGGCGTGTATCTGGTGGTAGGA-3′; | |
| | (SEQ ID NO: 14) | |
| marker | 5′-TCCTACCACCAGATACACGCCACAGTTTTTTTTTTATTA-3′; | 65 base |
| | (SEQ ID NO: 15) | |
| | 5′ CTGTGGCGTGTATCTGGTGGTAGGA-3′ | |
| | (SEQ ID NO :16) | |
| marker | 5′TTTTTTTTTTGATTA-3′-F: | 15 base |
| | (SEQ ID NO: 17) | |
| marker | 5′GAATA-3′-F; | 5 base |
| | (SEQ ID NO:18) | |

For comparison, the polarization of single-strand DNAs labeled on the 5′ (FIG. 1D, sub C) or 3′ (FIG. 1E, sub D) ends was also measured. The observed polarization signals were close to the values observed for sub B (both in the presence and absence of resolvase). In contrast, a higher polarization value was observed with a duplex molecule containing complete sequence complimentary to the labeled strand (FIG. 1F, sub E). This suggested mobility of the fluorescein-tagged single-stranded region prior to cleavage was reducing the polarization signal, likely due to rotational motion of single stranded DNA in sub B or stacking of fluorescein on the double-stranded DNA end in sub E.

Thus, it was believed that clamping down the single stranded ends of a splayed duplex substrate by engineering base complimentarity between the tips of the two single stranded segments of sub B would result in a higher polarization signal and a greater difference following cleavage. Thus, oligonucleotides were designed that, after annealing, would introduce a ten nucleotide bulge region flanked on one side by a long region of duplex DNA and on the other side by a 5 bp duplex region, or stem (FIG. 1G, sub F; “asymmetric bulge”). In a previous study, it was demonstrated that a substrate containing a central DNA bulge was efficiently cleaved at both sides of a bulge region (Culyba, et al, 2009, J Biol Chem 284:1190-1201). Sub F was fluorescein-labeled on the 3′-end of the stem region and tested with resolvase, demonstrating a 70 ml³ drop in FP. Evidently the 5 bp of pairing is sufficient for the duplex to be mostly paired in the starting substrate, but melt efficiently to allow release of the labeled strand after cleavage.

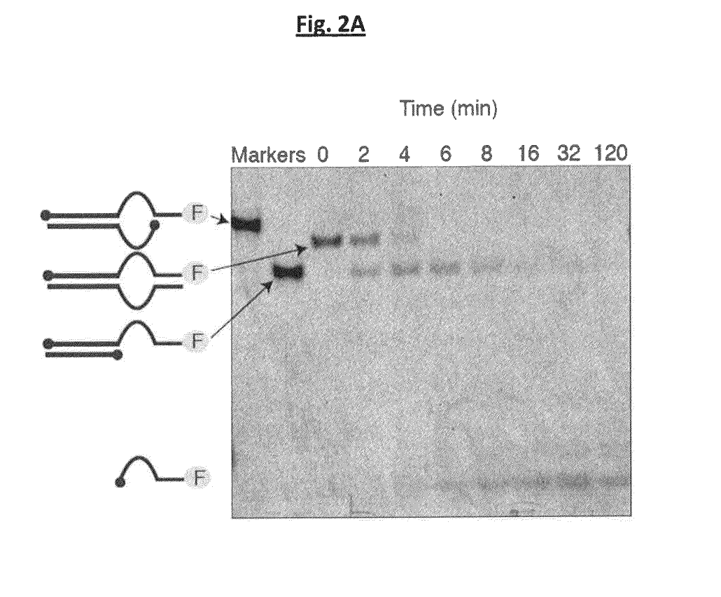

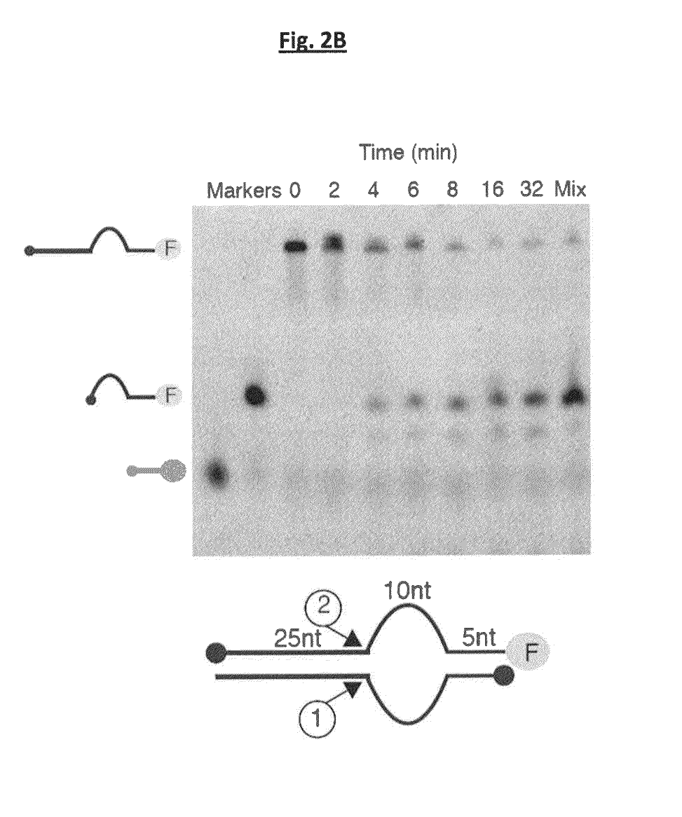

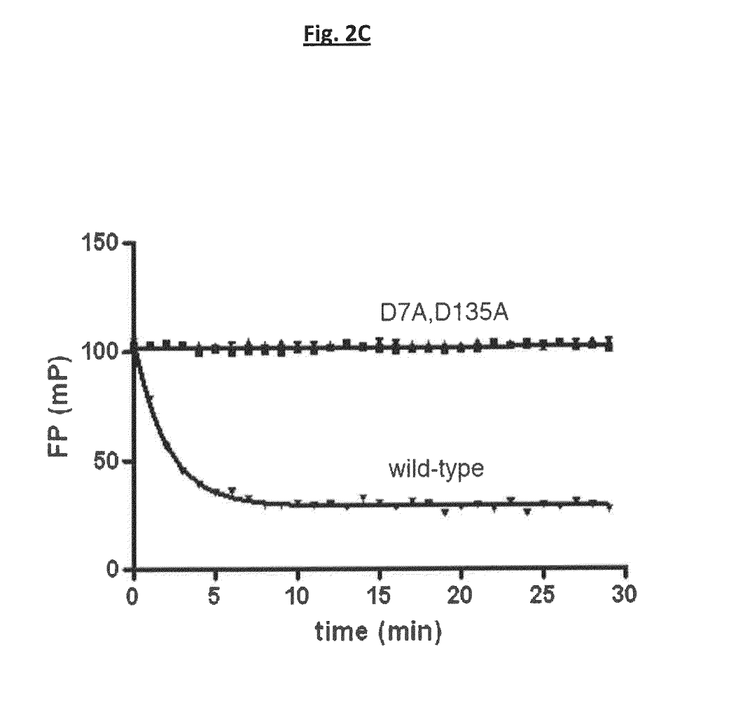

To characterize cleavage of this substrate more fully, the structure of reaction products was checked by gel electrophoresis (FIGS. 2A and 2B). Incubation with fowlpox resolvase yielded two cleavage products that appeared sequentially. Electrophoresis adjacent to synthetic oligonucleotides matching candidate cleavage products indicated sequential cleavage at the two single-to-double strand transitions shown at the bottom of FIG. 2B.

In the poxvirus replication cycle, this cleavage reaction may be important for resolving complex DNA structures generated during viral DNA replication. The poxvirus DNA polymerase is unusual for acting efficiently to fuse double strand broken ends containing terminal regions of homology, allowing use of the 3′ end at the double-strand break as a template for further elongation (Hamilton, et al., 2007, Nucleic Acids Res 35:143-151). Resolvase cleavage of an off-center bulged DNA at the sites mapped in FIG. 2 would thus generate a substrate for recombinational priming by the viral polymerase. The cleavage reaction described here would therefore allow restarting of DNA replication on molecules with complex unpaired regions.

A question that arises in any such study is whether the observed cleavage reaction is due to action of resolvase or cleavage by a low level contaminating protein in the resolvase preparation. To check this possibility, cleavage by FP resolvase was compared using two active site substitutions (D7A and D135A) previously shown to block cleavage by resolvase in vitro (Culyba, et al, 2009, J Biol Chem 284:1190-1201; Culyba, et al., 2010, J Mol Biol 399 (1): 182-95). These two mutants blocked all cleavage in the FP assay (FIG. 2C), confirming that fowlpox resolvase was responsible for the observed cleavage activity.

Using the FP assay, it was possible to show turnover of fowlpox resolvase at high substrate concentrations, allowing an investigation of the cleavage reaction using Michaelis-Menton kinetics, Vₘₐₓ was estimated to be 40 nM/min and K_{M} to be 226 nM (FIG. 5). The apparent turnover number k_{cat} for the off-center bulge substrate was 4 per minute, assuming that a dimer with two active sites cut a single double-stranded DNA on the two strands. The catalytic efficiency K_{cat}/K_{M} is thus 3×10⁵ M⁻¹ sec⁻¹. Cleavage of a Holliday junction by fowlpox resolvase in vitro was estimated at 13.8 per minute in a single turnover experiment, but only 0.24 per minute in a multiple turnover assay requiring completion of the catalytic cycle (Culyba, et al., 2009, J Biol Chem 284:1190-1201). Making the assumption that the rate limiting steps are the same for both substrates, the kinetic analysis suggests that cleavage is slower on the off-center bulge substrate, but this is associated with increased turnover compared to the Holliday junction. Data in FIG. 1G indicates that product is released efficiently after cleavage of the off-center bulge substrate, consistent with the idea that an increased rate of product release accounts for the acceleration of the reaction cycle.

To measure of the robustness of the assay, Z′- and Z-factor values, which, provide a measure of both the dynamic range and variance, were calculated. Z′- and Z-factor values range over −∞<Z≦1. Values greater than 0.5 indicate a large separation between the positive and negative controls with low variance suitable for high throughput screening (Zhang, et al., 1999, J Biomol Screen 4 (2):67-73). Using data obtained from five days of consecutive screening, Z′- and Z-factor values of 0.78 and 0.68, respectively were calculated.

Screening Compound Libraries Using the FP Assays

The off-center bulge substrate (sub F) was used to screen two small molecule libraries for inhibitors of resolvase DMA cleavage (Table 2). Library 1, from the National Screening Laboratory for the Regional Centers of Excellence in Biodefense and Emerging Infectious Disease (NSRB), contained a structurally diverse set of 130,540 compounds. Library 2 contained a structurally focused set of 2,788 compounds from Merck designed to target enzymes of the RNAse H superfamily, the majority of which contain metal chelating pharmacophores, which have been reported to be important in HTV integrase and RNAase H inhibitors (Hazuda, et al., 2000, Science, 287:646-650; Summa, et al., 2008, J Med Chem 51 (18):5843-55; Williams, et al., 2010, Bioorg Med Chem Lett 20:6754-6757).

Percent inhibition (PI) was calculated for each compound front the FP measurements at a single concentration. For Library 1, a compound was scored as a hit if its PI was greater than or equal to two standard deviations from the mean PI of all compound-containing wells. This hit definition corresponded to compounds with PI values ≧15% in the assay. 1,936 compounds meeting this hit criteria was identified, which corresponded to an overall hit rate of 1.5%, Using the same PI cut-off of 15% for Library 2, an overall hit rate of 45% for this library was obtained. Thus a 30-fold higher hit rate for Library 2 was obtained (Table 2). Since Library 2 contained primarily metal chelating compounds designed to target RNase H superfamily members, these data indicate that many of these metal chelating pharmacophores are inhibitors of poxvirus resolvase. The mechanism of inhibition of these metal chelators was not simple sequestration of the free Mg²⁺ cofactor away from the enzyme, because the Mg²⁻ concentration was 750-fold in excess of the inhibitor concentration.

TABLE 2
Results of high throughput screening using the off-center bulge substrate

| library | source | type of compounds | no. of molecules | hit rate |
|---|---|---|---|---|
| 1 | NSRB | diverse | 130,540 | 1.5% |
| 2 | Merck & Co. | metal-chelating | 2,787 | 45% |

Structure-Activity Analysts of 1-hydroxy-1,8-naphthyridin-2(1H)-one Inhibitors

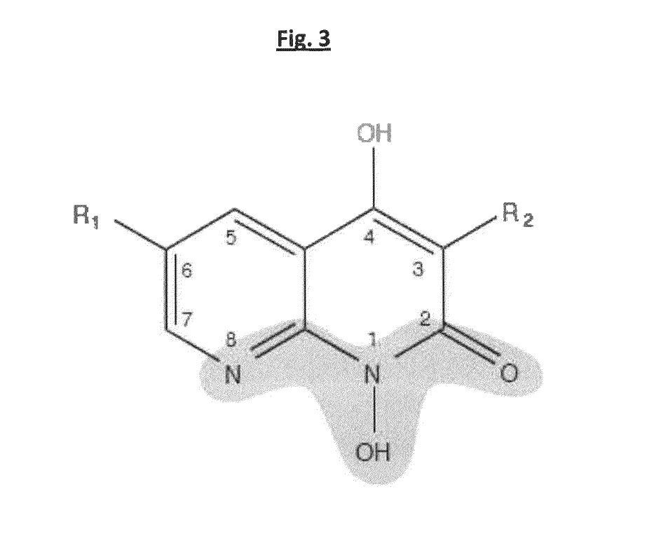

Based on initial results for resolvase inhibition in vitro, antiviral activity, and toxicity, 1-hydroxy-1,8-naphthyridin-2(1H)-ones were selected as a chemical series for follow up structure-activity (SAR) analysis (FIG. 3 and Table 3). The 1-hydroxy-1,8-naphthyridin-2(1H)-one compounds have recently been reported to inhibit HIV RNase H, an enzyme structurally related to poxvirus resolvase (Williams, et al., 2010, Bioorg Med Chem Lett 20:6754-6757). These compounds contain a potential metal chelating pharmacophore comprised of N8, the hydroxyl group at N1, and the carbonyl at position 2 in the naphthyridinone ring (FIG. 3). Compound 1 (Table 3), the initial hit from the library screen, showed an IC₅₀ value of 0.3 μM for resolvase inhibition in the FP assay. Resynthesis and re-testing of compound 1 confirmed that the activity was associated with the expected structure. To validate its activity, compound 1 was tested in resolvase cleavage reactions containing authentic Holliday junction substrates in vitro and found to show effective inhibition. Subsequent antiviral assays showed an IC₅₀ value of 4 μM. Cytotoxicity however was high, with LD₅₀ value of 13 μM.

Compounds 2-8 were synthesized in an effort to increase potency, reduce cytotoxicity, and begin to explore the SAR for this series of compounds. Since it is likely that, the resolvase inhibitory activity of compound 1 is due to its ability to both bind to the active site as well as chelate an active site divalent metal, modifications to the structure of compound 1 were designed to explore these aspects of its activity. Modifications to the groups at the 3 and 6 positions around the naphthyridinone ring were made to determine if these structural components were essential for binding. Modifications to the N-hydroxy group were made to test whether metal chelation is essential for activity. Some of the substitutions were chosen based on the structures of other hits in the initial library, and for synthetic accessibility. Compounds 2 and 4 were synthesized to investigate the tolerance for substitutions at the 6 position of the naphthrydinone ring. Both were slightly less potent in the FP assay than compound 1 and showed no reduction in cytotoxicity, To test the importance of the potential metal chelating pharmacophore, an analog with the N1 position substituted with a benzyloxy group was tested and this eradicated activity, supporting the idea that metal binding by the pharmacophore in FIG. 3 is essential for activity.

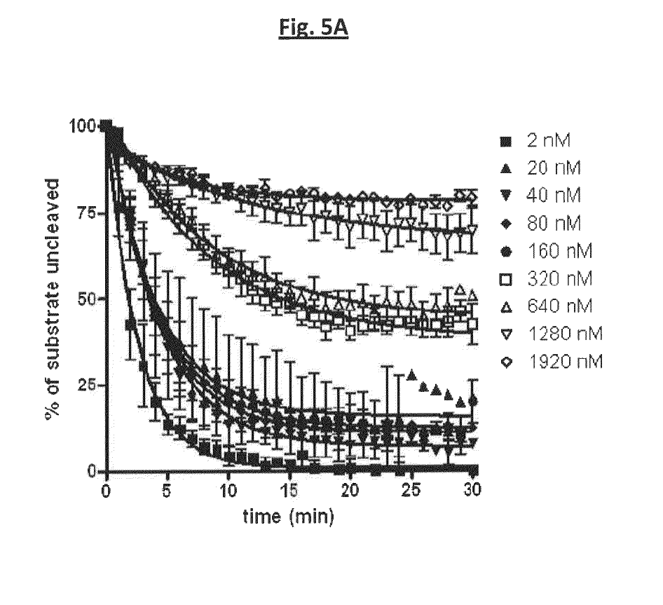

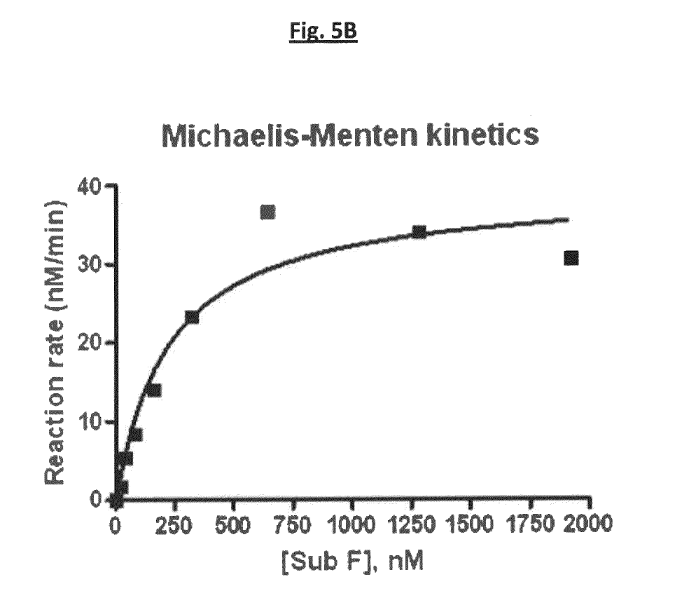

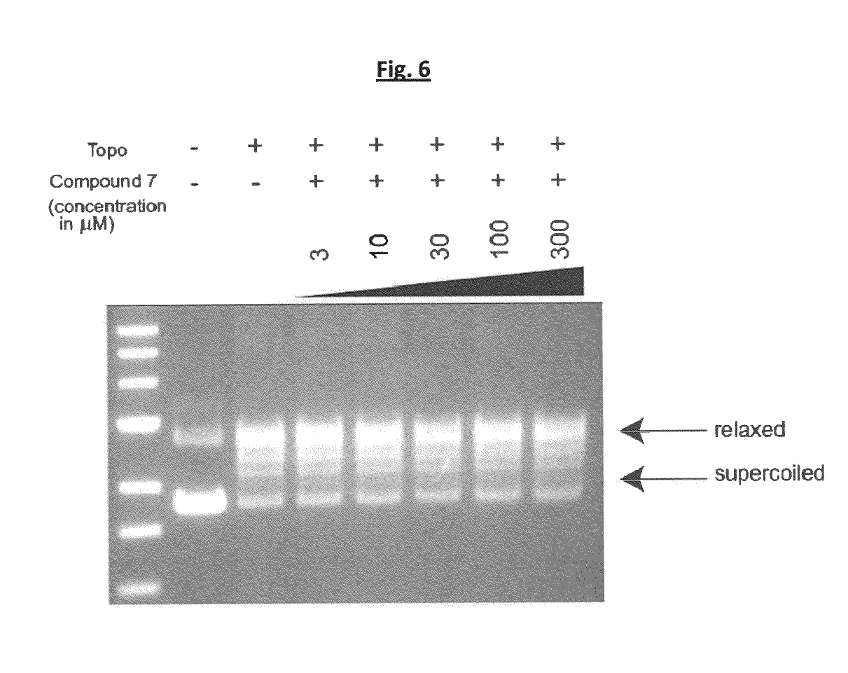

In an effort to improve the physiochemical properties of this series, the phenyl group at position 3 was substituted with an ethyl ester, yielding compounds 5-8. The ethyl ester was chosen based on its presence in active compounds in the initial library. This modification reduced the hydrophobicity (Table 3, see Log P values), which was higher in compounds 1-4 than is typical of inhibitors active its cells. Two pendant groups were compared at position 6 with the ethyl ester at position 3 (compounds 5 and 7). Compound 7 showed favorable characteristics. Initial tests of the TC₅₀ in the FP assay showed a slight increase over compound 1. Inhibition of Holliday junction cleavage was monitored using gel based assays and compound 7 was found to show a similar IC₅₀ (FIG. 4). As a test of specificity, activity of compound 7 was compared against the variola topoisomerase enzyme, and found to show no Inhibitory activity (FIG. 6), Importantly, though the IC₅₀ for viral infection was slightly increased, the LD₅₀ was greatly increased, so that the differential between antiviral activity and LD₅₀ was highest among the compounds studied (˜6-fold). Synthesis and testing of the N1 benzyloxy derivatives (6 and 8) confirmed for compounds 5 and 7 also that blocking the metal chelating pharmacophore abolished activity.

TABLE 3
SAR Analysis of Naphthyridone Resolvase Inhibitors

| | | | Antiviral | | |
|---|---|---|---|---|---|
| | | FP assay, | assay, | Cytotoxicity, | |
| Compd | Structure | IC50, uM | IC50, uM | LD50, uM | Log P |
| 1 | 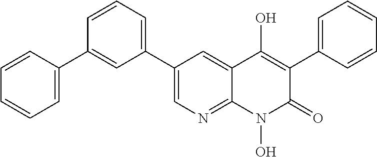 [SMILES: O=c1c(-c2ccccc2)c(O)c2cc(-c3cccc(-c4ccccc4)c3)cnc2n1O] | 0.3 ± 0.2 | 4 ± 1 | 13 ± 4 | 4.82 |
| 2 | 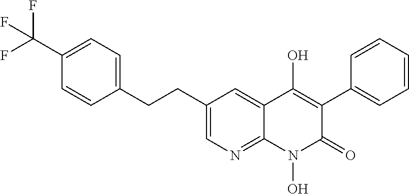 [SMILES: O=c1c(-c2ccccc2)c(O)c2cc(CCc3ccc(C(F)(F)F)cc3)cnc2n1O] | 3 ± 2 | 4 ± 1 | 15 ± 12 | 4.94 |
| 3 | 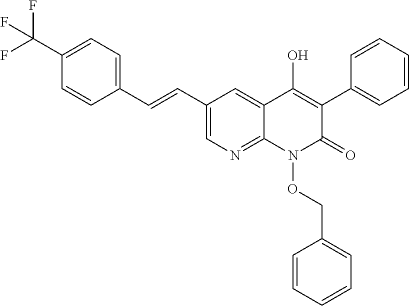 [SMILES: O=c1c(-c2ccccc2)c(O)c2cc(/C=C/c3ccc(C(F)(F)F)cc3)cnc2n1OCc1ccccc1] | >250 | >350 | nd | 6.85 |
| 4 | 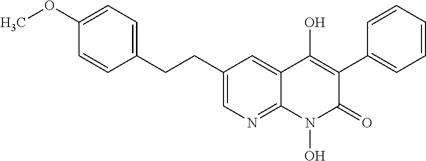 [SMILES: COc1ccc(CCc2cnc3c(c2)c(O)c(-c2ccccc2)c(=O)n3O)cc1] | 7 ± 1 | 11 ± 1 | 10 ± 1 | 3.90 |
| 5 | 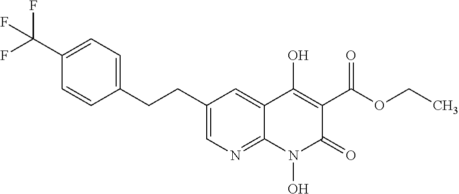 [SMILES: CCOC(=O)c1c(O)c2cc(CCc3ccc(C(F)(F)F)cc3)cnc2n(O)c1=O] | 14 ± 7 | 12 ± 1 | 20 ± 12 | 3.60 |
| 6 | 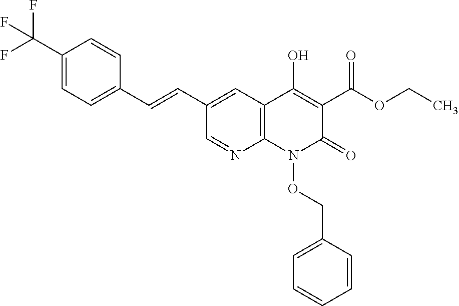 [SMILES: CCOC(=O)c1c(O)c2cc(/C=C/c3ccc(C(F)(F)F)cc3)cnc2n(OCc2ccccc2)c1=O] | >250 | >350 | nd | 5.50 |
| 7 | 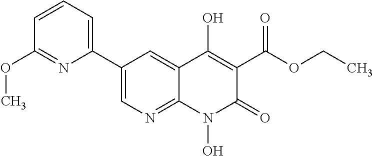 [SMILES: CCOC(=O)c1c(O)c2cc(-c3cccc(OC)n3)cnc2n(O)c1=O] | 11 ± 2 | 25 ± 16 | 156 ± 28 | 1.43 |
| 8 | 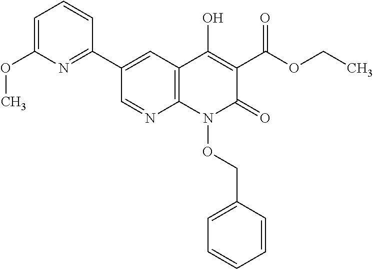 [SMILES: CCOC(=O)c1c(O)c2cc(-c3cccc(OC)n3)cnc2n(OCc2ccccc2)c1=O] | >250 | >350 | nd | 3.54 |

Identifying Inhibitors of Variola Virus

Resolvase enzymes that cleave DMA four-way (Holliday) junctions are required for poxvirus replication, but clinically useful inhibitors have not been developed. The present invention is based on the discovery that an assay based on fluorescence polarization (FP) can be used for high-throughput screening and mechanistic studies to evaluate resolvase cleavage activity. Initial analysis showed that cleavage of a fluorescently labeled Holliday junction substrate did not yield an appreciable change in FP. Without wishing to be bound by any particular theory, it is believed that the cleavage product did not have sufficiently increased mobility to yield a strong FP signal. Iterative optimization yielded a substrate with an off-center DNA bulge, which after cleavage released a labeled short stand and yielded a greatly reduced FP signal. Using this assay, 133,000 compounds were screened, identifying 1-hydroxy-1,8-naphthyridin-2(1H)-one compounds as inhibitors. Structure activity (SAR) studies revealed functional parallels to FDA-approved drugs targeting the related HIV integrase enzyme. Some 1-hydroxy-1,8-naphthyridin-2(1H)-one compounds showed anti-poxvirus activity.

Variola virus is a category A agent due to concerns about its possible use as a biological weapon. A Holliday junction resolvase is required for poxvirus replication. A bulged DNA substrate that reports resolvase cleavage activity efficiently using fluorescence polarization assays was developed for high throughput screening. Besides the practical utility, the activity on this substrate suggests a new function for resolvase that is believed to be important in linearizing branched DNA intermediates during poxvirus replication to restart DNA synthesis. Over 100,000 compounds were screened and it was found that 1-hydroxy-1,8-naphthyridin-2(1H)-one compounds constitute particularly active inhibitors. SAR analysis showed that potential metal chelating groups were important for inhibition, suggesting functional parallels with the FDA approved inhibitors of HIV integrase. One 1-hydroxy-1,8-naphthyridin-2(1H)-one (compound 7) showed antiviral activity at concentrations below cytotoxic levels. Thus these compounds and derivatives thereof can be used as inhibitors of variola virus for therapeutic and biodefense applications.

The disclosures of each and every patent, parent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (I), or a salt, solvate, or N-oxide thereof: 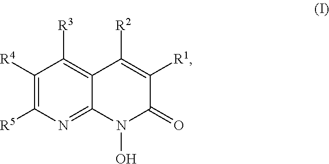
wherein in (I):
each occurrence of R¹ is independently selected from the group consisting of H, —C₁-C₆ alkyl, —C₁-C₆ alkenyl, —C₁-C₆ fluoroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), C₁-C₄ alkenyl-(aryl), and C₁-C₄ alkenyl-(heteroaryl), F, Cl, Br, I, —CN, —NO₂, —OR⁶, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;
wherein the alkyl, alkenyl, aryl, heteroaryl, heteroalkyl, cycloalkyl or heterocycloalkyl group is optionally substituted with 0-5 substituents, each of which is independently selected from the group consisting of —C₁-C₆ alkyl, —C₁-C₆ alkenyl, —C₁-C₆ fluoroalkyl, aryl, heteroaryl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), F, Cl, Br, I, —CN, —NO₂, —OR6, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —OC(═O)N(R⁶)₂, —NHC(═O)NH(R⁶), —NHC(50 O)R⁶, —NHC(═O)OR⁶, —C(OH)(R⁶)₂, and —C(NH₂)(R⁶)₂;
each occurrence of R² is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alky-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ and R² combine to form a (C₃-C₇)heterocycloalkyl group, a (C₃-C_{C}₇)cycloalkyl group, a (C₅-C₇)aryl group, or a (C₅-C₇)heteroaryl group optionally substituted with 0-2 R¹ groups;
each occurrence of R³, R⁴, and R⁵ is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O))N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups;
alternatively, R³ and R⁴ are combined to form a (C₃-C₇)heterocycloalkyl group, a (C₃-C₇)cycloalkyl group, a (C₅-C₇)aryl group, or a (C₅-C₇)heteroaryl group optionally substituted with 0-2 R¹ groups; and,
each occurrence of R⁶ is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl, or cycloalkyl group is optionally substituted with 0-5 R¹ groups.

2. The compound of claim 1, wherein in formula (I) R² is —OH and R³ and R⁵ are H.

3. The compound of claim 1, wherein in formula (I) R¹ is —C(═O)OCH₂CH₃, R² is —OH, and R³ and R⁵ are H.

4. The compound of claim 1, which is selected from the group consisting of:
6-([1,1′-biphenyl]-3-yl)-1,4-dihydroxy-3-phenyl-1,8-naphthyridin-2(1H)-one;
1,4-dihydroxy-3-phenyl-6-(4-(trifluoromethyl)phenethyl)-1,8-naphthyridin-2(1H)-one;
1,4-dihydroxy-6-(4-methoxyphenethyl)-3-phenyl-1,8-naphthyridin-2(1H)-one;
ethyl 1,4-dihydroxy-2-oxo-6-(4-(trifluoromethyl)phenethyl)-1,2-dihydro-1,8-naphthyridine-3-carboxylate;
ethyl 1,4-dihydroxy-6-(2-(6-methoxypyridin-2-yl)ethyl)-2-oxo-1,2-dihydro-1,8-naphthyridine-3-carboxylate;
a salt or solvate thereof, and any combinations thereof.

5. The compound of claim 1, wherein the compound is formulated as a pharmaceutical composition further comprising a pharmaceutically acceptable carrier.

6. A method of inhibiting poxvirus replication in a subject in need thereof, the method comprising administering to the subject a therapeutically effective amount of at least one compound of formula (I), or a salt, solvate, or N-oxide thereof: 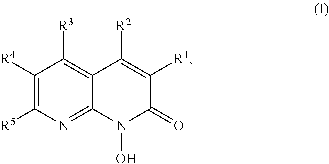
wherein in (I):
each occurrence of R¹ is independently selected from the group consisting of H, —C₁-C₆ alkyl, —C₁-C₆ alkenyl, —C₁-C₆ fluoroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), C₁-C₄ alkenyl-(aryl), and C₁-C₄ alkenyl-(heteroaryl), F, Cl, Br, I, —CN, —NO₂, —OR⁶, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;
wherein the alkyl, alkenyl, aryl, heteroaryl, heteroalkyl, cycloalkyl or heterocycloalkyl group is optionally substituted with 0-5 substituents, each of which is independently selected from the group consisting of —C₁-C₆ alkyl, —C₁-C₆ alkenyl, —C₁-C₆ fluoroalkyl, aryl, heteroaryl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), F, Cl, Br, I, —CN, —NO₂, —OR6, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —OC(═O)N(R⁶)₂, —NHC(═O)NH(R⁶), —NHC(50 O)R⁶, —NHC(═O)OR⁶, —C(OH)(R⁶)₂, and —C(NH₂)(R⁶)₂;
each occurrence of R² is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ and R² combine to form a (C₃-C₇)heterocycloalkyl group, a (C₃-C₇)cycloalkyl group, a (C₅-C₇)aryl group, or a (C₅-C₇)heteroaryl group optionally substituted with 0-2 R¹ groups;
each occurrence of R³, R⁴, and R⁵ is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O))N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups;
alternatively, R³ and R⁴ are combined to form a (C₃-C₇)heterocycloalkyl group, a (C₃-C₇)cycloalkyl group, a (C₅-C₇)aryl group, or a (C₅-C₇)heteroaryl group optionally substituted with 0-2 R¹ groups; and,
each occurrence of R⁶ is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl, or cycloalkyl group is optionally substituted with 0-5 R¹ groups.

7. The method of claim 6, wherein in formula (I) R² is —OH, and R³ and R⁵ are H.

8. The method of claim 6, wherein in formula (I) R¹ is —C(O)OCH₂CH₃, R² is —OH, and R³ and R⁵ are H.

9. The method of claim 6, wherein the compound of formula (I) is selected from the group consisting of:
6-([1,1′-biphenyl]-3-yl)-1,4-dihydroxy-3-phenyl-1,8-naphthyridin-2(1H)-one;
1,4-dihydroxy-3-phenyl-6-(4-(trifluoromethyl)phenethyl)-1,8-naphthyridin-2(1H)-one;
1,4-dihydroxy-6-(4-methoxyphenethyl)-3-phenyl-1,8-naphthyridin-2(1H)-one;
ethyl 1,4-dihydroxy-2-oxo-6-(4-(trifluoromethyl)phenethyl)-1,2-dihydro-1,8-naphthyridine-3-carboxylate;
ethyl 1,4-dihydroxy-6-(2-(6-methoxypyridin-2-yl)ethyl)-2-oxo-1,2-dihydro-1,8-naphthyridine-3-carboxylate;
a salt or solvate thereof, and any combinations thereof.

10. The method of claim 6, wherein the subject is a mammal.

11. The method of claim 10, wherein the mammal is a human.

12. A method of inhibiting poxvirus growth, the method comprising contacting the poxvirus with a growth inhibitory amount of at least one compound of formula (I), or a salt, solvate, or N-oxide thereof: 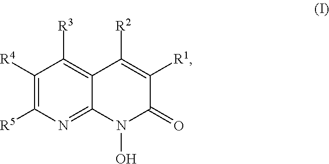
wherein in (I):
each occurrence of R¹ is independently selected from the group consisting of H, —C₁-C₆ alkyl, —C₁-C₆ alkenyl, —C₁-C₆ fluoroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), C₁-C₄ alkenyl-(aryl), and C₁-C₄ alkenyl-(heteroaryl), F, Cl, Br, I, —CN, —NO₂, —OR⁶, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;
wherein the alkyl, alkenyl, aryl, heteroaryl, heteroalkyl, cycloalkyl or heterocycloalkyl group is optionally substituted with 0-5 substituents, each of which is independently selected from the group consisting of —C₁-C₆ alkyl, —C₁-C₆ alkenyl, —C₁-C₆ fluoroalkyl, aryl, heteroaryl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), F, Cl, Br, I, —CN, —NO₂, —OR6, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —OC(═O)N(R⁶)₂, —NHC(═O)NH(R⁶), —NHC(50 O)R⁶, —NHC(═O)OR⁶, —C(OH)(R⁶)₂, and —C(NH₂)(R⁶)₂;
each occurrence of R² is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ and R² combine to form a (C₃-C₇)heterocycloalkyl group, a (C₃-C₇)cycloalkyl group, a (C₅-C₇)aryl group, or a (C₅-C₇)heteroaryl group optionally substituted with 0-2 R¹ groups;
each occurrence of R³, R⁴, and R⁵ is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O))N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups;
alternatively, R³ and R⁴ are combined to form a (C₃-C₇)heterocycloalkyl group, a (C₃-C₇)cycloalkyl group, a (C₅-C₇)aryl group, or a (C₅-C₇)heteroaryl group optionally substituted with 0-2 R¹ groups; and,
each occurrence of R⁶ is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl, or cycloalkyl group is optionally substituted with 0-5 R¹ groups.

13. The method of claim 12, wherein in formula (I) R² is —OH and R³ and R⁵ are H.

14. The method of claim 12, wherein in formula (I) R¹ is —C(O)OCH₂CH₃, R² is —OH, and R³ and R⁵ are H.

15. The method of claim 12, wherein the compound of formula (I) is selected from the group consisting of:
6-([1,1′-biphenyl]-3-yl)-1,4-dihydroxy-3-phenyl-1,8-naphthyridin-2(1H)-one;
1,4-dihydroxy-3-phenyl-6-(4-(trifluoromethyl)phenethyl)-1,8-naphthyridin-2(1H)-one;
1,4-dihydroxy-6-(4-methoxyphenethyl)-3-phenyl-1,8-naphthyridin-2(1H)-one;
ethyl 1,4-dihydroxy-2-oxo-6-(4-(trifluoromethyl)phenethyl)-1,2-dihydro-1,8-naphthyridine-3-carboxylate;
ethyl 1,4-dihydroxy-6-(2-(6-methoxypyridin-2-yl)ethyl)-2-oxo-1,2-dihydro-1,8-naphthyridine-3-carboxylate;
a salt or solvate thereof, and any combinations thereof.

16. The method of claim 12, wherein the subject is a mammal.

17. The method of claim 16, wherein the mammal is a human.

18. A method of treating a poxvirus infection in a subject in need thereof, the method comprising administering to the subject a therapeutically effective amount of at least one compound of formula (I), or a salt, solvate, or N-oxide thereof: 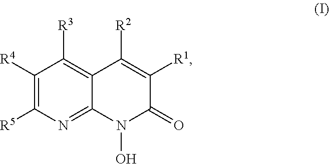
wherein in (I):
each occurrence of R¹ is independently selected from the group consisting of H, —C₁-C₆ alkyl, —C₁-C₆ alkenyl, —C₁-C₆ fluoroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), C₁-C₄ alkenyl-(aryl), and C₁-C₄ alkenyl-(heteroaryl), F, Cl, Br, I, —CN, —NO₂, —OR⁶, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, and —C(NH₂)(R⁶)₂;
wherein the alkyl, alkenyl, aryl, heteroaryl, heteroalkyl, cycloalkyl or heterocycloalkyl group is optionally substituted with 0-5 substituents, each of which is independently selected from the group consisting of —C₁-C₆ alkyl, —C₁-C₆ alkenyl, —C₁-C₆ fluoroalkyl, aryl, heteroaryl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), F, Cl, Br, I, —CN, —NO₂, —OR6, —SR⁶, —S(═O)R⁶, —S(═O)₂R⁶, —NHS(═O)₂R⁶, —C(═O)R⁶, —OC(═O)R⁶, —CO₂R⁶, —OCO₂R⁶, —CH(R⁶)₂, —N(R⁶)₂, —OC(═O)N(R⁶)₂, —NHC(═O)NH(R⁶), —NHC(50 O)R⁶, —NHC(═O)OR⁶, —C(OH)(R⁶)₂, and —C(NH₂)(R⁶)₂;
each occurrence of R² is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O)N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀ cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups, or R¹ and R² combine to form a (C₃-C₇)heterocycloalkyl group, a (C₃-C₇)cycloalkyl group, a (C₅-C₇)aryl group, or a (C₅-C₇)heteroaryl group optionally substituted with 0-2 R¹ groups;
each occurrence of R³, R⁴, and R⁵ is independently selected from the group consisting of H, —OR⁶, —C(═O)R⁶, —OC(═O)R⁶, —CO₂R⁶, —OCO₂R⁶, —CH(R⁶)₂, —N(R⁶)₂, —C(═O)N(R⁶)₂, —OC(═O))N(R⁶)₂, —NHC(═O)NH(R⁶), —NHC(═O)R⁶, —NHC(═O)OR⁶, —C(OH)(R⁶)₂, —C(NH₂)(R⁶)₂, C₁-C₆ alkyl, —C₁-C₆ alkenyl, —C₁-C₆ heteroalkyl, aryl, heteroaryl, cycloalkyl, heterocycloalkyl, —C₁-C₆ heteroalkyl, C₁-C₄ alkyl-(C₃-C₁₀cycloalkyl), C₁-C₄ alkyl-(C₂-C₁₀ heterocycloalkyl), C₁-C₄ alkyl-(aryl), and C₁-C₄ alkyl-(heteroaryl), wherein the alkyl, alkenyl, aryl, heteroaryl, heteroalkyl, cycloalkyl, or heterocycloalkyl group is optionally substituted with 0-5 R¹ groups;
alternatively, R³ and R⁴ are combined to form a (C₃-C₇)heterocycloalkyl group, a (C₃-C₇)cycloalkyl group, a (C₅-C₇)aryl group, or a (C₅-C₇)heteroaryl group optionally substituted with 0-2 R¹ groups; and,
each occurrence of R⁶ is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl, or cycloalkyl group is optionally substituted with 0-5 R¹ groups.

19. The method of claim 14, wherein in formula (I) R² is —OH and R³ and R⁵ are H.

20. The method of claim 14, wherein in formula (I) R¹ is —C(O)OCH₂CH₃, R² is —OH, and R³ and R⁵ are H.

21. The method of claim 14, wherein the compound of formula (I) is selected from the group consisting of:
6-([1,1′-biphenyl]-3-yl)-1,4-dihydroxy-3-phenyl-1,8-naphthyridin-2(1H)-one;
1,4-dihydroxy-3-phenyl-6-(4-(trifluoromethyl)phenethyl)-1,8-naphthyridin-2(1H)-one;
1,4-dihydroxy-6-(4-methoxyphenethyl)-3-phenyl-1,8-naphthyridin-2(1H)-one;
ethyl 1,4-dihydroxy-2-oxo-6-(4-(trifluoromethyl)phenethyl)-1,2-dihydro-1,8-naphthyridine-3-carboxylate;
ethyl 1,4-dihydroxy-6-(2-(6-methoxypyridin-2-yl)ethyl)-2-oxo-1,2-dihydro-1,8-naphthyridine-3-carboxylate;
a salt or solvate thereof, and any combinations thereof.

22. The method of claim 14, wherein the subject is a mammal.

23. The method of claim 22, wherein the mammal is a human.

24. A method of identifying a modulator of resolvase, the method comprising: (a) incubating a test substance with a protein having resolvase activity and a substrate of the protein: and (b) determining the resolvase activity of the protein in the presence of the test substance, wherein a change in resolvase activity of the protein as compared to a control indicates that the test substance is a modulator of resolvase activity.

25. The method of claim 24, wherein the protein having resolvase activity is resolvase.

26. The method of claim 24, wherein the substrate is labeled.

27. The method of claim 24, wherein the substrate is a nucleic acid molecule that represents a Holliday junction.

28. The method of claim 24, wherein the substrate is an off-center bulged nucleic acid molecule.

29. The method of claim 24, wherein the resolvase activity comprises cleaving the substrate, wherein detection of cleavage comprises characterizing the size of the cleaved substrate.


SEQUENCE LISTING (18 sequences)

SEQ ID NO: 1 - DNA, 40 bp, Artificial sequence
  Chemically synthesized
agcttctgtgcagcagctgctctcaactgcagtctagact

SEQ ID NO: 2 - DNA, 40 bp, Artificial sequence
  Chemically synthesized
agtctagactgcagttgagagcttgctaggacggatccct

SEQ ID NO: 3 - DNA, 40 bp, Artificial sequence
  Chemically synthesized
agggatccgtcctagcaagctttttgttttgattgcgagg

SEQ ID NO: 4 - DNA, 39 bp, Artificial sequence
  Chemically synthesized
cctcgcaatcaaaacaaaaagcagctgctgcacagaagc

SEQ ID NO: 5 - DNA, 40 bp, Artificial sequence
  Chemically synthesized
cctcgcaatcaaaacaaaaagcagctgctgcacagaagct

SEQ ID NO: 6 - DNA, 30 bp, Artificial sequence
  Chemically synthesized
gtcgtgctgctttttgttttgattgcgagg

SEQ ID NO: 7 - DNA, 40 bp, Artificial sequence
  Chemically synthesized
tcctaccaccagatacacgccacagttttttttttgatta

SEQ ID NO: 8 - DNA, 40 bp, Artificial sequence; identical to SEQ ID NO: 7
  Chemically synthesized

SEQ ID NO: 9 - DNA, 40 bp, Artificial sequence; identical to SEQ ID NO: 7
  Chemically synthesized

SEQ ID NO: 10 - DNA, 40 bp, Artificial sequence
  Chemically synthesized
taatcaaaaaaaaaactgtggcgtgtatctggtggtagga

SEQ ID NO: 11 - DNA, 40 bp, Artificial sequence; identical to SEQ ID NO: 7
  Chemically synthesized

SEQ ID NO: 12 - DNA, 40 bp, Artificial sequence
  Chemically synthesized
taatcttttttttttctgtggcgtgtatctggtggtagga

SEQ ID NO: 13 - DNA, 40 bp, Artificial sequence; identical to SEQ ID NO: 7
  Chemically synthesized

SEQ ID NO: 14 - DNA, 35 bp, Artificial sequence
  Chemically synthesized
ttttttttttctgtggcgtgtatctggtggtagga

SEQ ID NO: 15 - DNA, 40 bp, Artificial sequence; identical to SEQ ID NO: 7
  Chemically synthesized

SEQ ID NO: 16 - DNA, 25 bp, Artificial sequence
  Chemically synthesized
ctgtggcgtgtatctggtggtagga

SEQ ID NO: 17 - DNA, 15 bp, Artificial sequence
  Chemically synthesized
ttttttttttgatta

SEQ ID NO: 18 - DNA, 5 bp, Artificial sequence
  Chemically synthesized
gaata